(19) United States
(12) Patent Application Publication
(10) Pub. No.: US 2026/0137728 A1
(43) Pub. Date: May 21, 2026

(54) METHODS OF TREATING MALE HYPOGONADISM WITH HORMONE-PRODUCING ORGANOIDS

(71) Applicants: Craig S. ATWOOD, Fitchburg, WI (US); Sin-Ruow TEY, Madison, WI (US)

(72) Inventors: Craig S. ATWOOD, Fitchburg, WI (US); Sin-Ruow TEY, Madison, WI (US)

(21) Appl. No.: 19/445,265

(22) Filed: Jan. 9, 2026

Related U.S. Application Data
(63) Continuation-in-part of application No. 17/975,686, filed on Oct. 28, 2022.

(51) Int. Cl. A61K 35/52; A61P 15/08; C12N 5/071

(52) U.S. Cl. CPC A61K 35/52; A61P 15/08; C12N 5/0683; C12N 2501/165; C12N 2506/1353; C12N 2513/00

(57) ABSTRACT

A method of treating male hypogonadism in a subject in need thereof includes administering to the subject testicular organoids generated from isolated male progenitor cells. The method can be used to slow or halt the progression of age-related diseases, and increase healthspan and longevity, in hypogonadal males. The method can be used with subjects that have not or are not receiving testosterone replacement therapy. The method does not require that the subject is pre-treated with CG. The testicular organoid includes cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

### CROSS-REFERENCE TO RELATED APPLICATION

This application is a Continuation-in-part Application and claims priority to U.S. patent application Ser. No. 17/975,686 filed on Oct. 28, 2022, titled, “METHODS OF GENERATING HORMONE-PRODUCING ORGANOIDS AND REVERSING HYPOGONADISM,” the disclosure of which is incorporated herein by reference in its entirety.

### FIELD OF THE INVENTION

The present disclosure relates to hormone replacement. More particularly, the disclosure relates to a method of treating hypogonadism with hormone producing organoids.

### BACKGROUND OF THE INVENTION

Aging is regulated by reproductive hormones that act to promote growth and development early in life in order to achieve reproduction, maintain tissue function during reproductive life but later in life become dysregulated and drive senescence via altered cell cycle signaling (Bowen and Atwood 2004; Atwood and Bowen, 2011).

The principal hormones responsible for regulating reproductive function include the centrally and peripherally (primarily the gonads) produced hormones. In the human and many mammals, the centrally produced hormones include gonadotropin releasing hormone (GnRH) from the hypothalamus and the gonadotropins, luteinizing hormone (LH) and follicle stimulating hormone (FSH), from the pituitary. Peripherally produced hormones include estrogens, progestagens, androgens and inhibins that are primarily of gonadal origin while activins, follistatin and myostatin are produced in all tissues including the gonads (Carr 1998). The levels of each of these hormones are regulated by a complex feedback loop—GnRH secretion from the hypothalamus stimulates the anterior pituitary to secrete the gonadotropins, LH and FSH, which then bind to receptors in the gonads and stimulate oogenesis/spermatogenesis as well as sex steroid, anti-Müllerian hormone (AMH) and inhibin production (reviewed in Reichlin 1998). The sex steroids then feedback to the hypothalamus and pituitary, resulting in a decrease in gonadotropin secretion (reviewed in (Thorner, et al. 1998)). Activins, which are produced in many tissues, also stimulate gonadotropin secretion (Ling, et al. 1986; Vale, et al. 1986). Activin and myostatin stimulation of gonadotropin production (Ongaro et al., 2025) is inhibited by inhibins and follistatin, which are able to irreversibly bind to activins and prevent them from binding to their receptors (Cash, et al. 2012; DeKretser, et al. 2002; Gray, et al. 2002). Since inhibins are primarily produced in the gonads and their production is dependent on folliculogenesis/spermatogenesis (Knight and Glister 2001), they are intimately involved in the regulation of the hypothalamic-pituitary-gonadal (HPG) axis and are in fact a direct indicator of fertility.

The HPG axis is primarily regulated via the above mechanism. However, multiple other axes involving the hormones of the HPG axis have been found to exist throughout the body. Taking the brain as an example, these ‘mini-HPG’ axes include the Gonadal-Brain Neurosteroid Feedback Axis, the Pituitary-Brain Neurosteroid Feedback Axis, the Brain Neurosteroid Feedback Axis and the HPG-Extra-Hypothalamic Brain Neurosteroid Axis (see FIG. 5 in (Meethal, et al. 2009b)). In essence, the brain contains all the hormones, receptors, and cholesterol transport machinery of the HPG axis, and is regulated via feedback mechanisms. This mechanism may be of crucial importance following menopause and during andropause to compensate for the loss of circulating sex steroids (Meethal et al. 2009). This is supported by the findings that brain steroid levels are decreased in men and women with Alzheimer's disease (AD) compared with age-matched controls (Rosario, et al. 2011; Rosario, et al. 2004; Yue, et al. 2005). Similar interconnected feedback loops are expected for the hypothalamic-pituitary-adrenal axis, and other endocrine axes, and for the regulation of sex hormones in all other sex hormone producing tissues of the body including the placenta.

Gonadal produced sex hormones therefore regulate the concentration of HPG hormones in all tissues of the body. Likewise, hypothalamic and pituitary produced hormones can also influence the production of sex hormones in all tissues of the body.

Hormones produced by the gonads (gonadal hormones) include but are not limited to: sex steroids and their conjugated forms (17β-estradiol, 2-methoxyestradiol, estrone, estriol, pregnenolone, 17α-hydroxypregnenolone, 17α-hydroxyprogesterone, progesterone, testosterone, dihydrotestosterone, androstenedione, 1-andro stenediol, 4-androstenediol, 5-androstenediol, dehydroepiandrosterone, dihydroprogesterone, allopregnanlone, 17α-dihydroxyprogesterone, 17α-hydroxy allopregnanlone, 5α-androstenedione, androsterone, 5-androstenediol, 3α-androstenediol, 3β-androstenediol, α-triols, glucuronides, etiocholanolone, etiocholandione, etiocholandiol, 11-oxo-etiocholanolone, 11β-hydroxyandrosterone, 11β-hydroxy etiocholanolone, 16α-hydroxy dehydroepiandrosterone, 5-androstene-3β,16α,17β triol, 5-pregnene-3β,16α,17β triol, pregnanetriol, 11-oxo-pregnanetriol, pregnandione, pregnantrione, 17α-hydroxypregnanolone, pregnandiol, pregnandiol-20β), inhibins, activins, AMH, gonadotropin surge-attenuating factor, steroidogenic factor-1, liver receptor homolog-1, insulin-like growth factor, insulin, fibroblast growth factors, stem cell factor (Kit ligand), transforming growth factor-β, bone morphogenetic proteins (BMP), BMP4, and growth differentiation factors (GDF9, GDF11).

Reproductive hormones are required for the normal maintenance of all bodily tissues and their functions (Atwood and Bowen 2011). Receptors for reproductive tissues are found on almost every cell type in the human body (Atwood and Vadakkadath Meethal 2011a, b; Atwood, et al. 2005; Gallego, et al. 2010; Gallego, et al. 2009; Meethal, et al. 2009a; Meethal, et al. 2005; Vadakkadath Meethal and Atwood 2005; Wilson, et al. 2006). Reproductive hormones signal via these receptors to drive cell division (e.g. luteinizing hormone, follicle-stimulating hormone, gonadotropin-releasing hormone) while certain other reproductive hormones allow cell cycle completion and promote cell differentiation (i.e. cell specification and function, e.g. sex steroids, activins, inhibins, follistatin, myostatin). When these hormones are in balance there is appropriate cell division and differentiation for the replacement of cells lost during normal tissue maintenance. This turnover of cells in tissues allows normal tissue function.

The dysregulation of the reproductive axis (HPG axis; FIG. 1), otherwise known as reproductive endocrine dyscrasia, leads to altered (dyotic) signaling to all cells in the body. Since the hormones of the reproductive HPG axis are involved in cellular mitogenesis and differentiation, this dyotic signaling leads to aberrant cell cycle signaling, cellular dysregulation and dysfunction, and/or cell death in all tissues of the body, eventually leading to organ failure and death (Atwood and Bowen 2011; Bowen and Atwood 2004).

Reproductive endocrine dyscrasia that leads to dysregulation of the mini-HPG axes present in tissues throughout the body leads to dyotic signaling, aberrant cell cycle signaling, cellular dysregulation and dysfunction, and/or cell death in those tissues of the body, eventually leading to organ failure and death.

The age-related dysregulation of the HPG axis in women (FIG. 1A) is a direct result of the loss of ovarian follicles in the female (Bowen and Atwood 2004); primordial follicle numbers fall from ˜1,000,000 at birth to a few thousand non-functional follicles at menopause (Wallace and Kelsey 2010). Granulosa and thecal cells of the follicle produce sex hormones (e.g. estrogens, progestagens, testosterone, inhibins, AMH) that regulate the release of the ovum (ovulation), but these hormones also negatively feedback on the HP to maintain the axis in equilibrium. With the loss of follicles, gonadal sex hormones are no longer synthesized and this results in the loss of negative feedback on the hypothalamus and pituitary, leading to the dysregulation of the HPG axis, and menopause. The timing of menopause is variable (average age is approximately 51 in the USA) due to the variance in the total number of follicles that a female is born with together with the rate of folicular atresia throughout the female reproductive period. Endocrine dyscrasia also occurs following surgical removal of the ovaries (oophorectomy) and in certain disease states and conditions.

The age-related dysregulation of the HPG axis in men (FIG. 1B), which is more gradual but starts earlier at around 30 years of age (FIG. 1B) is a direct result of the loss of testicular Leydig, Sertoli, spermatogonial stem cells, and other gonadal cells (Belanger, et al. 1994). Leydig and Sertoli cells are the male equivalent of thecal and granulosa cells; they are the testicular support cells that produce androgens, inhibins, AMH, estradiol, glial cell line-derived neurotrophic factor and other hormones required for spermatogenesis. Each year after the age of 30, there is a ˜1-2% decrease in testosterone production by the testes that corresponds to a ˜1-2% decrease in Leydig cell number (Belanger et al. 1994; Tserotas and Merino 1998). Endocrine dyscrasia also occurs following surgical removal of the testes and in certain disease states and conditions.

After menopause, and during andropause, the decrease in the production of gonadal sex hormones such as inhibins (Reichlin 1998) leads to an increase in bioavailable activins (Gray, et al. 2002), thereby increasing hypothalamic GnRH and pituitary gonadotropin production (MacConell, et al. 1999; Schwall, et al. 1988; Weiss, et al. 1993). Likewise, the concurrent decrease in gonadal sex steroid production results in a loss of hypothalamic/pituitary feedback inhibition and also stimulates GnRH and gonadotropin production (Carr 1998). In women the loss of this negative feedback by estrogens and inhibins (Couzinet and Schaison 1993) results in a three- to four-fold and a four- to eighteen-fold increase in the concentrations of serum LH and FSH, respectively (Chakravarti, et al. 1976). Likewise, men also experience a greater than two-fold, and three-fold, increase in LH and FSH, respectively as their reproductive function deteriorates (Neaves, et al. 1984).

This reproductive endocrine dyscrasia, associated with menopause, andropause and hypogonadism, is associated with the development of numerous symptoms, and senescent and age-related diseases and conditions. The HPG axis also is dysregulated in a number of primary congenital, primary acquired, secondary congenital and secondary acquired conditions and diseases.

Maintaining the HPG axis in balance decreases the risk of developing senescent and age-related disease and extends longevity in humans. For example, menopause at a later age reduces the risk of morbidity and mortality; the incidence of cardiovascular disease, calcifications in the aorta, atherosclerosis, osteoarthritis, cognitive decline, osteoporosis, bone fractures and certain cancers is reduced (see: Atwood and Bowen 2011; Ossewaarde, et al. 2005; Yonker, et al. 2011; Lizneva et al., 2018; Liu et al., 2020; Eun et al., 2022; Butler et al., 2024; FIG. 2). Conversely, early reproductive endocrine dyscrasia occurring naturally or induced by bilateral oophorectomy in premenopausal women, increases risk of morbidity and mortality (Rocca, et al. 2006); the incidence of dementia, cognitive decline, stroke, fatal and non-fatal coronary heart disease, Parkinsonism, osteoporosis, hip fracture, lung cancer, depression and anxiety are increased (e.g. Gleason, et al. 2005; Parker and Manson 2009; Rocca et al. 2006; Rocca, et al. 2008; Rocca, et al. 2012; Lizneva et al., 2018; Liu et al., 2020; Eun et al., 2022; Butler et al., 2024).

Manipulation of the HPG axis in model organisms provides strong evidence for a direct link between HPG axis balance and longevity (Arantes-Oliveira, et al. 2002). In Mammalia (Mus musculus), re-establishment of the negative feedback loops in the HPG axis of post-reproductive mice (11 months of age) following transplantation of reproductively viable ovaries from young mice (3 months of age) has been demonstrated to extend lifespan by up to 40% (Cargill, et al. 2003; Mason, et al. 2009). In Actinopterygii, certain long-lived species (including Sebastes aleutianus (Rougheye rockfish; >205 years) and Sebastes alutus (Pacific ocean perch; 98 years)) maintain their HPG axis in balance by maintaining a constant number of ovarian follicles (de Bruin, et al. 2004). In Insecta (Drosophila melanogaster) heterozygous for inactivating mutations in the ligand binding domain or DNA binding domain of ecdysone receptor there is a robust (typically 20-50%) lifespan-extension (Simon, et al. 2003). In Secernentea (Caenorhabditis elegans), suppression of GnRH receptor signaling significantly decreases reproduction 46% and prolongs lifespan 15% (23% at lower temperature) compared with wild-type worms (Vadakkadath Meethal, et al. 2006; Vadakkadath Meethal, Bowen and Atwood, unpublished data).

Strategies to completely rebalance the HPG axis have not been attempted. Supplemental add-back of one or more steroid hormones or drugs provides partial rebalancing of the HPG axis with decreases in menopausal and andropausal symptoms and reduces the risk of morbidity and decreases mortality (reviewed in Paganini-Hill, et al. 2006 and 2018). However, rebalancing this axis using one or two hormones or drugs is insufficient to completely rebalance the entire axis (Atwood and Bowen, 2011), does not replicate the minute-to-minute, hour-to-hour and day-to-day needs of the individual, and comes with increased risk of vascular and other issues.

Creation of testes in vitro in the form of testicular organoids possessing the major testicular cell types allows normal autocrine and paracrine signaling between these cell types, and coupled with endocrine signaling from extra-gonadal tissues, allows optimal production of all sex hormones. Testicular development and function are the culmination of a complex process of autocrine, paracrine and endocrine interactions between multiple cell types (reviewed in Smith et al., 2015). For example, macrophages and Leydig cells share an intricate physical and physiological relationship; macrophage ablation has revealed that this relationship is essential for Leydig cell development and regeneration, and for normal sex steroid synthesis. There is also an intricate cross talk between germ cells and Sertoli cells, while the development and survival of the adult Leydig cell population is fundamentally dependent upon Sertoli cells. Thus, the normal development of the testes requires a coordinated differentiation process involving all testicular cell types, while the normal production of the multitude of sex steroids and sex protein hormones also requires the presence of complex feed-forward and feed-back signaling between the various testicular cell types.

Male hypogonadism is a syndrome defined by inadequate production of gonadal testosterone (T), inhibins, anti-Müllerian hormone, and the multitude of other hormones produced by the testes (Bhasin et al., 2018; Jayasena et al., 2022). It is generally accompanied with concurrent increases in hypothalamic/pituitary hormones (GnRH, LH and FSH). Together, these hormonal changes are known as reproductive endocrine dyscrasia. Testicular hormones regulate spermatogenesis and libido, and, together with other hormones of the hypothalamic-pituitary-gonadal (HPG) axis, support fetal and pubertal growth and development and the maintenance of tissue health in adulthood (reviewed in Kaufman & Vermeulen, 2005; Kaufman & T'Sjoen, 2019; Bhasin, 2022). However, the dysregulation of this axis ultimately leads to tissue degeneration, disease and death (Bowen and Atwood, 2004). Male hypogonadism reflects defects within the testes (primary) or in the hypothalamic-pituitary organs (secondary).

Aging-related primary hypogonadism is commonly termed late-onset hypogonadism, or testosterone deficiency syndrome (TDS). In the US, an estimated 4-5 million men carry a clinical diagnosis of severe primary hypogonadism, while all men over the age of ˜50 suffer from undiagnosed TDS, the diminished capacity to produce the upward of 50 different testicular hormones that include sex steroids and protein sex hormones (Boston University Sexual Medicine; AUA Guideline overview). Hypogonadism substantially impairs quality of life and adversely affects multiple organ systems (reviewed in Kaufman & T'Sjoen, 2019; Bhasin et al., 2018). Beyond classic symptoms—reduced libido, infertility, low energy, cognitive difficulties, and increased adiposity—chronic hypogonadism is strongly associated with age-related diseases such as Alzheimer's disease, hypertension, obesity, type 2 diabetes, and osteoporosis (Kaufman & Vermeulen, 2005; Kaufman & T'Sjoen, 2019). Clinical trials demonstrate that testosterone therapy can improve cognition, sexual function, bone mineral density, body composition, and quality of life (Testosterone Trials; Snyder et al., 2016; Snyder et al., 2017; Ponce et al., 2018). However, benefits and risks for cardiovascular and prostate outcomes remain uncertain (TRAVERSE trial; AUA guidance and subsequent reviews).

Late-onset hypogonadism reflects loss and dysfunction of Leydig and Sertoli cells, which produce >20 other sex steroids including testosterone, inhibins, activins, anti-Müllerian hormone, and related reproductive protein hormones (Kaufman & T'Sjoen, 2019; Matzkin et al., 2021). Pathologic hallmarks of testicular aging include a spectrum of seminiferous tubule lesions—from reduced but present spermatogenesis to complete sclerosis—along with Sertoli-cell vacuolation, Leydig-cell apoptosis, and expansion of extracellular spaces (Paniagua et al., 1987; Nie et al., 2022). Beginning after age 30, testicular production of testosterone and inhibins declines by ˜1-2% per year, paralleling a ˜1-2% annual reduction in Leydig-cell number (Harman et al., 2001; Neaves et al., 1985). Declines in testosterone and inhibins A/B reduce negative feedback on the hypothalamus and pituitary, leading to increased GnRH output and elevated LH and FSH concentrations (Neaves et al., 1984; Vadakkadath and Atwood, 2005; Bhasin, 2022).

Congenital primary hypogonadism affects ˜300,000 cases in the US and includes Klinefelter syndrome (47,XXY), Noonan syndrome, defects in testosterone synthesis, and androgen-resistance states; acquired causes include cryptorchidism, chemo-/radiotherapy-induced testicular damage, bilateral torsion, orchitis, orchidectomy, and various acute or chronic diseases (Bhasin et al., 2018; Medscape Klinefelter's overview, 2019). Secondary (hypogonadotropic) hypogonadism, characterized by low testosterone with low gonadotropins, occurs in ˜5% of men; ˜0.1% of cases are congenital, with the remainder acquired due to infiltrative or infectious pituitary disease, hyperprolactinemia, head trauma, cranial irradiation, and related conditions (Fraietta et al., 2013; Tajar et al., 2010; Bhasin et al., 2018).

The standard therapy for hypogonadism is testosterone supplementation (Bhasin et al., 2018). It only partially addresses hypogonadism, as it does not replace the multitude of sex steroids and protein sex hormones such as inhibin A/B, activin A, anti-Müllerian hormone, kit ligand, etc. (Kaufman & T'Sjoen, 2019). These protein hormones lack practical pharmacologic replacements and cannot be administered like testosterone (e.g., orally, transdermally, or subcutaneously). Testosterone monotherapy also carries significant risks; for example, failure to restore inhibins—tumor suppressors—may raise carcinogenic potential (Matzuk et al., 1992). Additional complications of testosterone monotherapy include: (1) uncertainty about the appropriate physiological target, as most patients do not know their “age-25” testosterone level; (2) risk of supraphysiologic dosing; (3) failure to mimic natural hour-to-hour and day-to-day hormonal dynamics; (4) suppression of endogenous testosterone; (5) testicular atrophy from gonadotropin suppression; and (6) infertility and other potential risks (e.g., cancer, thromboembolism) (AUA Guideline; Patel et al., 2018). In short, testosterone therapy alleviates symptoms but does not restore physiologic testicular endocrine function—much like insulin therapy improves diabetes management without resolving the underlying disease.

What is needed is an alternative to testosterone therapy for treating hypogonadism.

### SUMMARY OF THE INVENTION

This disclosure is based on the discovery that based on pre-clinical testing in accepted rat models it is possible to reverse male hypogonadism and increase longevity using hormone producing organoids.

Embodiments of the present disclosure pertain to methods of creating organoids composed of the major cell types required for normal testicular endocrine function.

Embodiments of the disclosure pertain to methods of treating male hypogonadism and/or increasing longevity with hormone producing organoids. In certain embodiments, the methods involve administering to a subject testicular organoids generated from isolated male progenitor cells. In some embodiments of the methods, the subject has not been pre-treated with a hormone such as human chorionic gonadotropin.

Embodiments of the present disclosure pertain to methods of creating testicular organoids composed of Sertoli cells, spermatogonial stem cells, myoid cells, Leydig cells, macrophages and endothelial cells.

Embodiments of the present disclosure pertain to methods of creating ovarian organoids (follicles) composed of granulosa cells, oocytes, myoid cells, thecal cells, macrophages and endothelial cells.

An embodiment of the invention pertains to the production of sex protein hormones and sex steroids by the testicular or/and ovarian organoids.

Embodiments of the present disclosure are capable of improving integration and/or the function of gonadal organoids (testicular or ovarian) introduced to a subject, at least to some extent.

An embodiment of the invention pertains to a method of improving integration of gonadal organoids introduced to a subject. The method includes the steps of pre-treating the subject with a chorionic gonadotrophin (CG), administering a therapeutically effective amount of the gonadal organoids to the subject, and administering the CG to the subject periodically over time to cause a fluctuation of a concentration of the CG in the subject.

Another embodiment of the invention relates to a method of improving functionality of gonadal organoids introduced to a subject. The method includes the steps of pre-treating the subject with a chorionic gonadotrophin (CG), administering a therapeutically effective amount of the gonadal organoids to the subject, and administering the CG to the subject periodically over time to cause a fluctuation of a concentration of the CG in the subject.

Another embodiment of the invention pertains to a method of treating a patient. In this method, a hypothalamic-pituitary-gonadal (HPG) axis of the patient in need thereof is rebalanced by administering a therapeutically effective amount of at least one donor cell.

Another embodiment of the invention relates to a method of reducing endocrine dyscrasia (dyosis) in a patient. In this method, a hypothalamic-pituitary-gonadal (HPG) axis of the patient in need thereof is rebalanced by administering a therapeutically effective amount of at least one gonadal organoid.

Yet another embodiment of the invention pertains to a method of reducing rejection in a patient in need of a tissue-specific stem cell transplant. In this method, a hypothalamic-pituitary-gonadal (HPG) axis of the patient in need thereof is rebalanced by administering a therapeutically effective amount of at least one gonadal organoid followed by administering a stem cell that is tissue-specific to the patient.

Yet another embodiment of the invention relates to a method of preventing or slowing dyosis in a patient. In this method, a therapeutically effective amount of at least one physiological agent that regulates or increases the production of hormones produced by the gonads is administered to a patient.

Another embodiment of the invention pertains to a method of treating male hypogonadism in a subject in need thereof comprising administering to the subject testicular organoids generated from isolated male progenitor cells. In certain embodiments, the subject is not being administered testosterone replacement therapy. In other embodiments, the subject is not pre-treated with chorionic gonadotrophin (CG). In some embodiments, the hypogonadism is testosterone deficiency syndrome (TDS) or congenital primary hypogonadism. The subject is a male mammal such as a human. In certain embodiments, the human is 50 years old or older. The method can include injection of the organoids.

Yet another embodiment of the invention pertains to a method of increasing health span or longevity in a male subject in need thereof comprising administering to the subject testicular organoids generated from isolated male progenitor cells. In certain embodiments, the subject is not pre-treated with chorionic gonadotrophin (CG). The subject is a male mammal such as a human. The method can include injection of the organoids.

A further embodiment of the invention pertains to a method of rebalancing the HPG axis in a male subject in need thereof comprising administering to the subject testicular organoids generated from isolated male progenitor cells. In certain embodiments, the subject is not pre-treated with chorionic gonadotrophin (CG). The subject is a male mammal such as a human. In certain embodiments, the human is 50 years old or older. The method can include injection of the organoids.

In some embodiment, the male progenitor cells are isolated from the subject. In other embodiments, the testicular organoid includes cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR. In certain embodiments, the subject is not receiving testosterone supplementation. In some embodiment, the method restores hormone balance. In other embodiments, the method further includes pre-treating the subject with a chorionic gonadotrophin (CG). In preferred embodiments, the method does not further include pre-treating the subject with a chorionic gonadotrophin (CG).

In certain embodiments, the method further includes generating the testicular organoids by expanding isolated male progenitor cells, such as e.g. adult-derived or umbilical cord-derived mesenchymal stem cells, adipose-derived stem cells (ADSC), embryonic stem cells, or induced-pluripotent stem cells, generating a spheroid under dynamic culture conditions, and culturing the spheroid under dynamic culture conditions to generate the testicular organoid. In some embodiment, the method includes culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

Yet another embodiment of the invention pertains to a method of generating a testicular organoid, which includes: (a) generating a spheroid from isolated male progenitor cells under dynamic conditions; and (b) culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid. In some embodiments, the testicular organoid includes cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR. In some embodiments, the testicular organoid includes at least 5-30% cells expressing PDGF-Rα, Cyp11A1, 3β-HSD, 17β-HSD, and LHCGR, about 5-50% of cells expressing α-SMA and CD11b, about 5-50% of cells expressing MHC CII and CD64, at least 5-30% of cells expressing Sox9, AR, inhibin β-B, and FSHR, and about 5-30% of cells expressing VEGF and VEGFR2, and cells expressing about 3-15% GFRA1. The progenitor cells can be adult-derived or umbilical cord-derived mesenchymal stem cells, or adipose-derived stem cells (ADSC) or embryonic-derived stem cells. In some embodiments, the method includes culturing the cells for at least 7 days or for about 14 days to about 27 days. In other embodiments, the method further includes culturing the progenitor cells in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1 illustrates the changes in hypothalamic-pituitary-gonadal hormones before and following menopause in women (A) and before and during andropause in men (B).

FIG. 2 illustrates the adjusted survival probability curves for women aged between 50 and 90 whose last menstrual period occurred at 40, 45, 50, or 55 years of age (Yonker et al. 2011).

FIG. 3 illustrates primordial follicle number is closely correlated with longevity between species. Controlling for ovulation rate, for every additional 10,000 primordial follicles an animal is born with, there is an associated ˜0.8% increase in longevity (Atwood et al., unpublished data).

FIG. 4 illustrates a method of gonadal organoid formation from stem cells for injection into a subject for restoring HPG axis balance.

FIG. 5 illustrates treatment of rBM-MSCs with CDM-3.4 increases markers over 27 days of six testicular cell types including spermatogonial stem cells (SSC), and vascular (endothelial), Leydig, myoid, macrophage (Mø) and Sertoli cells.

FIG. 6 illustrates injection of castrated hCG-treated Fisher rats with gonadal organoids at 14 or 27 days of differentiation increases circulating DHEA concentrations.

FIG. 7 illustrates injection of castrated hCG-treated Fisher rats with gonadal organoids at 14 or 27 days of differentiation increases circulating testosterone concentrations.

FIG. 8 illustrates injection of castrated hCG-treated Fisher rats with gonadal organoids at 14 or 27 days of differentiation increases circulating 17β-estradiol concentrations.

FIG. 9 illustrates a schematic for the testing protocol in Example 14A. The protocol includes obtaining mesenchymal stem cells from male Fischer rats, differentiating these stem cells into organoids and subcutaneously injecting the organoids into castrated Fischer rats.

FIG. 10 illustrates bright-field micrographs depicting rat organoid formation from cell aggregation to differentiation. The six bright-field micrographs show progressive spheroid formation at days relative to differentiation start (d0): d-5 (24 h with H1152), d-4, d0, d3, d7, and d10. At d-5, mostly single cells and small clusters are present; by d-4, loose micro-aggregates appear; by d0, compact spheroids have formed, and the addition of CDM-3 media defines the start of differentiation. From d3 to d10, spheroids enlarge and round with increasingly smooth borders. Scale bars=400 μm.

FIG. 11 illustrates flow-cytometric profiling of CDM-3 rat organoids during early differentiation. Bars depict mean values from a representative batch, showing percent positive cells at d0 (black), d5 (magenta), and d10 (teal) after gating on singlets and Live/Dead-negative events with FMO controls. Markers are grouped by lineage left-to-right: testicular macrophages (MHC II, CD64); Leydig (PDGF-Rα, CYP11A1, 3β-HSD, 17β-HSD, LHR); myoid (α-SMA, CD11b, MYH11); Sertoli (SOX9, AR, inhibin-βB, FSHR, AMH); SSC (PLZF, GFRA1, SSEA-4); angiogenesis (VEGF, VEGFR2). Data acquired using Attune NxT cytometer with ≥50,000 events per marker.

FIG. 12 illustrates plasma steroid trajectories after rat organoid implantation. Time courses for testosterone, progesterone, DHEA, and 17β-estradiol are shown from pre-castration and post-castration baselines through 18 weeks post-injection. The vertical dotted line marks the time of injection. X-axes: time post-injection (weeks); Y-axes: hormone concentration (units labeled per panel). Symbols represent mean±SEM; bars depict mean values from a representative experiment.

FIG. 13 illustrates lifespan after a single dose of rHPOs. Left panel illustrates Kaplan-Meier survival for male Fischer 344 rats: castrated untreated, castrated treated with rBM-MSC organoids, and an intact reference curve reproduced from literature (reference only). Vertical annotations indicate castration and injection times. Right panel shows per-group lifespan distributions with mean±SEM and summary table. *p=0.012.

The drawings presented are intended solely for the purpose of illustration and therefore, are neither desired nor intended to limit the subject matter of the disclosure to any or all of the exact details of construction shown, except insofar as they may be deemed essential to the claims.

### DETAILED DESCRIPTION OF THE PREFERRED EMBODIMENTS

The present disclosure relates to a method of maintaining in balance or rebalancing, the hypothalamic-pituitary-gonadal (HPG) axis and preventing or reversing hypogonadism and accompanying symptoms and diseases. In particular, this disclosure relates to treating male hypogonadism using hormone-producing organoids. More particularly, embodiments of the present invention relate to a method for slowing, preventing or delaying senescence, preventing or treating a disease associated with senescence, and for increasing longevity. This is achieved by delivering donor cells into the human or animal body to increase the production and secretion of sex hormones into the circulation to levels near young adult reproductive levels, thereby reinitiating negative feedback on the hypothalamus and pituitary to rebalance the HPG axis hormone synthesis and secretion to levels near young adult reproductive levels. This in effect prevents dyotic (death) signaling that results from the dysregulation of the HPG axis. This will prevent and treat hypogonadism, prevent and treat symptoms associated with female reproductive endocrine dyscrasia and symptoms associated with male reproductive endocrine dyscrasia, and prevent or delay the onset of age-related diseases and extend longevity.

It has been determined that longevity between species is directly correlated with the number of ovarian primordial follicles that an animal is born with together with the rate at which they are used (FIG. 3; Atwood et al.). Therefore, maintaining endocrine gonadal cells to prevent reproductive endocrine dyscrasia offsets senescent and age-related disease and increase longevity (see Example 14).

Preventing the dysregulation of the HPG axis by repopulating the ovaries with follicular cells, and the testes with Leydig, Sertoli and other support cells, will prevent hypogonadism, symptoms thereof, and delay the onset of age-related diseases and extend longevity. As shown below, it has been established that such a repopulating can be achieved by using hormone-producing organoids that obtained from pluripotent stem cell, such as, for example, mesenchymal stem cells.

Restoring the HPG axis to balance (young adult reproductive levels) by repopulating the ovaries with follicular cells, and the testes with Leydig, Sertoli and other support cells, will reverse hypogonadism, symptoms thereof, and delay the onset of age-related diseases and extend longevity.

Extra-gonadal injections of donor cells capable of rebalancing the HPG axis also will reverse hypogonadism, symptoms thereof, and delay the onset of age-related diseases and extend longevity.

Embodiments of the present invention relates to a method for preventing or reversing hypogonadism and increasing longevity by decreasing or preventing dyotic signaling via the rebalancing or maintenance of the HPG axis; by altering the blood and/or tissue levels, production, function, or activity of HPG axis hormones to be near the blood and/or tissue levels, production, function, or activity occurring during fetal life or at or around the height of the subject's reproductive function, in a subject, by administering donor cells that regulate the blood levels, production, function, or activity of any hormone produced by the gonads. This is a method for slowing, preventing or delaying senescence or treating or preventing a disease associated with senescence.

For clarity of disclosure, and not by way of limitation, the detailed description of the invention is divided into subsections that describe or illustrate certain features, embodiments, or applications of the present invention.

The disclosure provides for methods of generating hormone-producing organoids and treatment methods that rely on these hormone-producing organoids.

The disclosure also provides for methods of generating hormone-producing spheroids and treatment methods that rely on these hormone-producing spheroids.

### Definitions

By “height of the subject's reproductive function” is meant that time (usually between 18-35 years of age) when subjects are most fertile and reproductive hormones are at their optimal for reproduction. “Subjects” may include humans or animals.

By “donor cell” is meant any cell or group of cells (e.g., aggregates, spheroids, organoids, or the like) that are undifferentiated (pluripotent, totipotent, multipotent, or induced pluripotent stem cells), differentiated and/or dedifferentiated that are to be transplanted into the body, or any organ of the body, so as to 1) repopulate that organ or that is transplanted for the purpose of reestablishing the function of that organ or inducing a new function, or 2) act as a discrete organ residing in another organ or tissue for the purpose of restoring a bodily function. The cells can be from the host organism (autologous cells) or from a donor organism (allogeneic cells).

The term “mesenchymal stem cells” (MSCs) refers to multipotent stromal cells that can differentiate into a variety of cell types, including osteoblasts (bone cells), chondrocytes (cartilage cells), myocytes (muscle cells) and adipocytes (fat cells).

The term “adipose-derived stem cells” refers to a subset of MSCs obtained from adipose tissues with multipotential differentiation and self-renewal properties.

Most cells are “terminally differentiated,” meaning they no longer possess the ability to complete the “cell cycle,” the process by which cells undergo chromosome replication and division to create two new daughter cells (Jacobsen 1991). Although terminally differentiated cells may be able to enter the cell cycle, they are unable to complete the process and usually undergo apoptosis (i.e., cell death) (Multani, et al. 2000). This can lead to cell loss and the development of a number of different diseases, including, but not limited to, Alzheimer's disease, Parkinson's disease, vascular dementia, frontal-temporal dementia, stroke, neoplasia, coronary heart disease, chronic obstructive pulmonary disease, osteoporosis, arthritis, diabetes mellitus II, etc. For example, evidence suggests that vascular smooth muscle cell proliferation leads to atherosclerosis (Lusis 2000). Cancers may result when terminally differentiated cells lose the protective ability to apoptose and are able to complete the cell cycle, resulting in abnormally increased cell proliferation (Hahn and Meyerson 2001).

Dysregulated HPG axis, dysregulated HPG signaling, endocrine dyscrasia and reproductive endocrine dyscrasia are used interchangeably to indicate the HPG axis (or other hormone axes) is not in balance, whether this be a result of aging, genetics or acquired.

“Dyosis” is the process by which dysregulated cell cycle signaling drives the biochemical and functional changes associated with senescence. Dysregulated cell cycle signaling, caused by altered mitogenic and differentiative stimuli, contributes to the development of the above diseases. By “dysregulated cell cycle signaling” is meant an increased frequency or rate of cells entering into the cell cycle, and/or inability of cells to complete the cell cycle. By “increased mitogenic stimulus” is meant an increase in the blood levels, production, function or activity of a mitogenic stimulus or a decrease in the blood levels, production, function, or activity of an anti-mitogenic stimulus. By “mitogenic stimulus” is meant a compound that acts as an impetus for cells to enter into the cell cycle, including, but not limited to, gonadotropin releasing hormone (GnRH), luteinizing hormone (LH), human chorionic gonadotropin (hCG), follicle-stimulating hormone (FSH), and activins. Throughout this application, the terms “upregulation of the cell cycle”, “increased mitogenic stimulus” and “dysregulated cell cycle signaling” and “dyotic signaling” are used interchangeably. By “abnormally increased proliferation” is meant increased proliferation of cells that interferes with the normal function of the tissues and/or threatens the life or health of the subject.

The term, “hCG”, “drug”, or “active agent” as used herein refers to any suitable therapeutic agent. Drugs/active agents for use in the present technology include human chorionic gonadotropic (hCG), human luteinizing hormone (hLH), follicle-stimulating hormone or combinations of two or more thereof. In any embodiment, the drugs/active agents for use in the present technology includes hCG. Naturally occurring hCG and hLH are heterodimeric glycoprotein hormones, each comprising an α subunit and a β subunit. Each subunit consists of a single polypeptide chain which are non-covalently bound to each other. The α subunit polypeptide is common to both hCG and LH, whereas the β subunits differ in sequence from each other. Each of the hormones may exist as a mixture of isoforms, including differentially glycosylated isoforms. In certain embodiment, where the subject is a non-human mammal, the CG and/or LH polypeptides used in the present methods refer to the CG and/or LH isoforms native to the subject species and may include deletional, insertional, or substitutional mutants of such native CG and/or LH. In any embodiments, the CG and/or LH may also be a functional agonist of a native mammalian LHCG receptor.

“Effective amount” refers to the amount of compound (here, the drug) or composition required to produce a desired effect. Hence, an effective amount of a compound or composition of the present technology in the context of treatment (i.e., “a therapeutically effective amount”) refers to an amount of the compound or composition that alleviates, in whole or in part, symptoms associated with a disorder or disease (e.g., reverse or diminish the cognitive impairment/induce cognitive recovery), or slows or halts further progression or worsening of those symptoms. In the context of prevention, an effective amount prevents at least partially or provides prophylaxis for the disease or disorder in a subject at risk for developing the disease or disorder. One example of an effective amount includes amounts or dosages that yield acceptable toxicity and bioavailability levels for therapeutic (pharmaceutical) use. Determining a therapeutically effective amount of a compound described herein for treating a particular disorder or disease is well within the skill in the art in view of the present disclosure.

As used herein, a “subject” or “patient” is a mammal, such as a cat, dog, ungulate, rodent or primate. In any embodiments, the subject is a human. The term “subject” and “patient” can be used interchangeably.

“Treating” or “treatment” within the context of the present technology, means an alleviation, in whole or in part, of symptoms associated with a disorder or disease, or slowing, or halting of further progression or worsening of those symptoms. As a non-limiting example of treatment, a subject can be successfully treated for hormonal dysregulation if, after receiving through administration an effective or therapeutically effective amount of one or more drugs, biologics or compositions described herein, the subject shows observable and/or measurable induced cognitive recovery. Treatment, as defined herein, of a subject, including a human being, is subject to medical aid with the object of improving the subject's condition, directly or indirectly. Treatment typically refers to the administration of an effective amount of a drug or composition containing a drug such as hCG as described herein.

“Menopause” is the cessation of a woman's reproductive ability due to the cessation of the primary functions of the ovaries. These primary functions include the inability to ovulate and release hormones which leads to reproductive (HPG axis) endocrine dyscrasia. Menopause is usually a natural change; it typically occurs in women in midlife, during their late 40s or early 50s, signaling the end of the fertile phase of a woman's life. However, in some women menopause can occur earlier or later. Menopause can also occur from premature ovarian failure or following bilateral oophorectomy (surgical menopause). Premature ovarian failure may be attributed to autoimmune disorders, thyroid disease, diabetes mellitus, polycystic ovary syndrome, being a carrier of the fragile X syndrome gene, chemotherapy and radiotherapy. The causes of the majority of spontaneous cases of premature ovarian failure are unknown, i.e. it is generally idiopathic. In the present disclosure, we are focusing on rebalancing all HPG hormones and not just estrogens and progesterone.

Premature ovarian failure, also known as premature ovarian insufficiency, primary ovarian insufficiency, premature menopause, hypergonadotropic hypogonadism, is the loss of function of the ovaries before age 40.

“Andropause” is the gradual cessation of testicular function leading to endocrine dyscrasia and occurs in men starting at around 30 years of age (Atwood and Bowen 2011). Total testosterone declines by 0.9-1.6% per year after age 30, while free testosterone declines at a higher rate due to the concurrent increase in sex hormone binding globulin at an average rate of 1.3% per year, thereby compounding the effect of depleted total testosterone (Atwood and Bowen 2011; Feldman, et al. 2002; Gapstur, et al. 2002; Harman, et al. 2001; Liu, et al. 2007; T'Sjoen, et al. 2005; Travison, et al. 2007a; Travison, et al. 2007b). In the present disclosure, we are focusing on rebalancing all the hormones and not just testosterone.

“Hypogonadism” is a diminished functional activity of the gonads, pituitary or hypothalamus—that results in diminished sex hormone biosynthesis. Primary congenital forms of hypogonadism that impact ovarian function include being a carrier of the fragile X syndrome gene, Turner syndrome, Noonan syndrome, XY females with SRY gene-immunity, inborn errors of estrogen or progesterone synthesis, and estrogen or progestagen resistant states. Primary acquired forms of hypogonadism that impact ovarian function include autoimmune disorders, hysterectomy, oophorectomy, tuberculosis of the genital tract, thyroid disease, diabetes mellitus, prolonged GnRH therapy, chemotherapy, radiotherapy and various acute and chronic systemic disease. Primary congenital forms of hypogonadism that impact testicular function include Klinefelter's syndrome, Noonan's syndrome, inborn errors of testosterone synthesis, and androgen resistant states. Primary acquired forms of hypogonadism that impact testicular function include cryptorchidism (undescended testes), bilateral torsion, orchitis, orchidectomy (castration), gonadal toxins, including radiotherapy and chemotherapy, and various acute and chronic diseases.

Hypogonadotropic hypogonadism, also known as secondary or central hypogonadism, as well as gonadotropin-releasing hormone deficiency or gonadotropin deficiency (GD), is a condition which is characterized by hypogonadism due to an impaired secretion of gonadotropins, including follicle-stimulating hormone (FSH) and luteinizing hormone (LH), by the pituitary gland in the brain, and in turn decreased gonadotropin levels and a resultant lack of sex steroid production.

Secondary congenital forms of hypogonadism (hypogonadotropic hypogonadism) that impact testicular and/or ovarian function include Kallman syndrome, isolated GnRH deficiency, isolated LH deficiency, Prader-Willi syndrome, Turner syndrome, and Laurence-Moon-Biedl syndrome. Secondary acquired forms of hypogonadism that impact testicular and/or ovarian function include pituitary tumors and infarct, trauma, mumps, traumatic brain injury, children born to mothers who had ingested the endocrine disruptor diethylstilbestrol, opioid induced androgen deficiency (resulting from the prolonged use of opioid class drugs, e.g. morphine, oxycodone, methadone, fentanyl, hydromorphone), anabolic steroid-induced hypogonadism craniopharyngioma, hyperprolactemia (1° & 2°), hemochromatosis and neurosarcoid.

Late-onset hypogonadism (LOH, also referred to as age-associated testosterone deficiency syndrome (TDS)) is a clinical and biochemical syndrome associated with advancing age and characterized by symptoms and a deficiency in serum testosterone and inhibin levels (below the young healthy male reference range); ISA, ISSAM, EAU, EAA and ASA recommendations, 2009, International Society for Sexual Medicine (ISSM), 2010).

Testosterone deficiency syndrome (TDS) includes both severe hypogonadism described above and milder forms of hypogonadism whereby circulating testosterone concentrations have fallen by more than 30%. The most common form of TDS is age-related, driven by the yearly loss of 1-2% of key testicular cells (Sertoli and Leydig cells) that produce inhibins and testosterone, respectively. This loss starts at around 30 years of age, such that by age 50, gonadal hormone levels have fallen by 20-40%. Since all men start to lose gonadal cells around age 30, all men over 30 can be defined as having TDS, albeit with few symptoms (see below) until levels fall by 20-40%.

The terms andropause, hypogonadism, testosterone deficiency syndrome, late-onset hypogonadism, male menopause, hypergonadotropic hypogonadism, male climacteric, androgen decline in the aging male (ADAM), and aging male syndrome all involve age-related, congenital or acquired reproductive endocrine dyscrasia in the male, and are used interchangeably in terms of HPG axis dysregulation (male reproductive endocrine dyscrasia).

The terms menopause, hypogonadism, premature ovarian failure, premature ovarian insufficiency, primary ovarian insufficiency, premature menopause, hypergonadotropic hypogonadism, all involve age-related, congenital or acquired reproductive endocrine dyscrasia in the female, and are used interchangeably in terms of HPG axis dysregulation (female reproductive endocrine dyscrasia).

Hypogonadism, hypergonadotropic hypogonadism, andropause, menopause and related conditions may result in significant detriment in the quality of life and adversely affect the function of multiple organ systems. These conditions are associated with numerous symptoms and diseases of aging.

Symptoms associated with female reproductive endocrine dyscrasia include vasomotor instability (hot flushes, night sweats, cold flashes), migraines, rapid heartbeat, and dysfunctional bleeding (Arakane, et al. 2011; Dreisler, et al. 2013; Freeman, et al. 2014; Freeman, et al. 2009; Freeman, et al. 2008; Freeman, et al. 2007; Llaneza, et al. 2011; Llaneza, et al. 2012; Monterrosa-Castro, et al. 2013; Monterrosa-Castro, et al. 2012; Ornat, et al. 2013; Perez-Lopez, et al. 2012; Pien, et al. 2008). Other symptoms include:
1. Urogenital (vaginal) atrophy—thinning of the membranes of the vulva, the vagina, the cervix, and also the outer urinary tract, along with considerable shrinking and loss in elasticity of all of the outer and inner genital areas, itching, dryness, watery discharge, urinary frequency, urinary continence, urinary urgency, increased susceptibility to inflammation and infection, for example vaginal candidiasis, and urinary tract infections.
2. Skeletal—back pain, joint pain, osteoarthritis, muscle pain, sarcopenia, osteopenia, and the risk of osteoporosis gradually developing over time, height loss.
3. Skin, soft tissue—breast atrophy, breast tenderness with and without swelling, decreased elasticity of the skin, formication (itching, tingling, burning, pins, and needles, or sensation of ants crawling), skin thinning and becoming drier, alopecia, increased weight gain, body fat and BMI, and frailty.
4. Psychological—depression and/or anxiety, fatigue, irritability, memory loss, and problems with concentration, mood disturbance, sleep disturbances, poor or light sleep, insomnia, and daytime sleepiness.
5. Sexual—dyspareunia, decreased libido and orgasm.

Symptoms associated with male reproductive endocrine dyscrasia include (Lincoln 2001; Sternbach 1998) vasomotor instability (hot flushes, night sweats, cold flashes), migraines, and rapid heartbeat. Other symptoms include:
1. Urogenital atrophy—small or shrinking testes, infertility; urinary frequency, urinary continence, urinary urgency.
2. Skeletal—back pain, joint pain, osteoarthritis, muscle pain, sarcopenia, osteopenia, and the risk of osteoporosis gradually developing over time, height loss.
3. Skin, soft tissue—breast discomfort, gynecomastia, decreased elasticity of the skin, formication (itching, tingling, burning, pins, and needles, or sensation of ants crawling), skin thinning and becoming drier, alopecia, increased weight gain, body fat and BMI, and frailty.
4. Psychological—depression and/or anxiety, dysthymia, fatigue, decreased energy, motivation, self-confidence and work performance, irritability, hypersensitivity, anger, memory loss, and problems with concentration, mood disturbance, sleep disturbances, poor or light sleep, insomnia, and daytime sleepiness.
5. Sexual—decreased libido and activity, erectile dysfunction (decreased spontaneous erections), delayed sexual development.

In this specification, by “senescence” is meant any change in the function of an organism, or any of its tissues, that occurs concomitantly with a decline in reproductive function after the period of greatest reproductive function, which in humans typically corresponds to about 18 to 35 years of age. By “disease associated with senescence” is meant any disease, disorder, degeneration, tissue loss, or other unhealthy or abnormal condition caused by, linked to, or otherwise associated with senescence. Examples of diseases associated with senescence include, but are not limited to, atherosclerosis, brain cancer (including but are not limited to neuroma, anaplastic astrocytoma, neuroblastoma, glioma, glioblastoma multiforme, astrocytoma, meningioma, pituitary adenoma, primary CNS lymphoma, medulloblastoma, ependymoma, sarcoma, oligodendroglioma, medulloblastoma, spinal cord tumor, and schwannoma), polyps of the colon and colorectal cancer, myeloproliferative diseases (including but not limited to Hodgkin's disease, multiple myeloma, lymphoma, transient myeloproliferative disorder (TMD) (also known as transient myeloproliferative syndrome), congenital transient leukemia, congenital leukemoid reaction, transient leukaemoid proliferation, transient abnormal myelopoiesis, acute myeloid leukemia (AML), acute megakaryoblastic leukemia (AMKL) (also known as erythro-megakaryoblastic leukaemia); common B-lineage acute lymphoblastic leukemia (ALL), polycythemia, thrombocythemia, myelodysplastic syndromes, myelofibrosis, hypereosinophilic syndrome (HES), chronic lymphocytic leukemia, prolymphocytic leukemia, hairy-cell leukemia, chronic myelogenous leukemia, other leukemias, and other myelogenous cancers), osteoarthritis, osteoporosis, neoplasms, cataracts, macular degeneration, hearing loss, stroke, periodontal disease, osteopenia, peripheral neuropathy, COPD, hypertension, type 2 diabetes, sarcopenia, hypertension, primary pulmonary hypertension, congestive heart failure, left ventricular hypertrophy, cardiac valvular disease, dementia, Alzheimer's disease, mild cognitive impairment, frontotemporal dementia, esophagitis, esophageal stricture, gastroparesis, chronic pancreatitis, hypercholesterolemia, hypertriglyceridemia, cirrhosis of the liver, liver disease, Wilson's disease, kidney disease, hepatitis, cholelithiasis, cholecystitis, ulcerative colitis, inflammatory bowel disease, Crohn's disease, fibromyalgia, obesity, renal failure, proteinuria, gout, hyperuricemia, membranous nephropathy, polyarteritis nodosa, polymyalgia rheumatica, rheumatoid arthritis, progressive systemic sclerosis, spinal stenosis, spinal cord injury, migraine headaches, depression, major depression, anxiety, hair loss, baldness, male pattern baldness, sarcoidosis, Wegener granulomatosis, amyloidosis, dermatomyositis, graft versus host disease, systemic lupus erythematosus, seborrheic dermatitis, psoriasiform eczematous dermatitis, papulosquamous eczematous dermatitis, psoriasis, seborrheic keratosis, anagen effluvium, dysphagia, Barrett esophagus, achalasia, Chagas disease, facial neuropathy, trigeminal neuralgia, carpal tunnel syndrome, mitochondrial myopathies and encephalopathies, myasthenia gravis, traumatic brain injury, astrocytomas, oligodendrogliomas, meningiomas, schwannomas, pituitary adenomas, pineocytoma and pineoblastoma, primary central nervous system lymphoma, medulloblastomas, spinal cord tumors, paraneoplastic syndromes, anoxic encephalopathics, multiple sclerosis, Duchenne's muscular dystrophy, muscular dystrophy, transverse myelitis, Parkinson's disease, Lewy body disease, squamous cell carcinoma of the lung, adenocarcinoma of the lung, large cell carcinoma of the lung, small cell carcinoma of the lung, esophageal cancer, gastric cancer, pancreatic cancer, hepatocellular cancer, gallbladder carcinomas, colorectal cancer, Hodgkin's disease, non-Hodgkin's lymphoma, follicular lymphoma, small lymphocytic lymphoma, mantle cell lymphoma, marginal zone lymphoma, diffuse large cell lymphoma, Burkitt's and Burkitt's-like lymphoma, lymphoblastic lymphoma, peripheral T-cell lymphoma, large cell (T-cell and null) anaplastic lymphoma, primary anaplastic lymphoma, multiple myeloma, Ewing's sarcoma, chondrosarcomas, osteosarcomas, renal cell carcinoma, bladder carcinoma, testicular carcinoma, seminoma, nonseminoma, squamous cell carcinoma of the head and neck, salivary gland tumors, pneumoconioses, asbestosis, silicosis, coal worker's pneumoconiosis, berylliosis, malignant diffuse infiltrative lung disease, disease caused by pulmonary lymphangitic carcinomatosis, disease caused by alveolar cell carcinoma, chronic diffuse infiltrative lung disease of unknown etiology, sarcoidosis, idiophatic pulmonary fibrosis, desquamative interstitial pneumonia/respiratory bronchiolitis, interstitial lung disease, acute interstitial pneumonia, lymphocytic interstitial pneumonia, nonspecific interstitial pneumonia/fibrosis, bronchiolitis obliterans, Sjogren syndrome, mixed connective tissue disease, eosinophilic granuloma of the lung, allergic granulomatosis and anguitis, hypereosinophilic syndrome, osteoarthritis, spinal arthritis, ankylosing spondylitis, reactive arthritis (formerly known as Reiter syndrome), psoriatic arthritis, enteropathic arthritis, juvenile spondyloarthropathy, acne-associated arthritis, SAPHO (synovitis, acne, pustulosis, hyperostosis, osteitis) syndrome, Whipple disease, Paget's disease of bone, osteomalaci, decreased muscle mass, decreased skin elasticity, increased rhytids, thinning of skin, decreased scalp hair growth, loss of subcutaneous collagen, decreased immune function, autoimmune disorders, decreased lung function, loss of arterial elasticity, urinary incontinence, degenerative disk disease, loss of renal function, brain damage associated with acute brain injury, tissue injury associated with acute tissue injury, Addison's disease, reduced reproductive capacity, reduced follicular number, reduced sperm motility, reduced semen volume, reduced sperm count and varicose veins.

Dysregulated cell cycle signaling (dyotic signaling), as occurs when the HPG axis becomes dysregulated with menopause or during andropause, and as occurs with congenital or acquired conditions described above, can lead to the aberrant re-entry of stem cells or post-mitotic (terminally differentiated) cells into the cell cycle. Such signaling leads to cell dysfunction and degeneration and altered cellular hormone levels that are not conducive to the normal functioning of tissues and the health of the subject. Such altered cell cycle signaling has been reported to lead to numerous diseases associated with senescence and aging, including but not limited to:
1. Cognitive decline/dementia. Elevated gonadotropins modulate the processing of amyloid-ß protein precursor and amyloid-ß deposition, promotes tau phosphorylation and drives aberrant neuronal cell division (Bowen, et al. 2004; McPhie, et al. 2003); Atwood and Bowen, 2015; Bowen et al., 2015; Xiong et al., 2022).
2. Decreased cerebrovascular function: Elevated gonadotropins increase the permeability of the blood-brain barrier, a precursor to stroke (Wilson, et al. 2008).
3. Osteoporosis. Elevated follicle-stimulating hormone increases the ratio of osteoclasts to osteoblasts, resulting in bone resorption and osteoporosis (Sun, et al. 2006).
4. Sarcopenia (Tey and Suzuki, personal communication).
5. Osteoarthritis (Huan et al., 2021).
6. Renal function (Li et al., 2021).
7. Immune dysregulation. Dyotic signaling leads to suppressed immune function.
8. Cancer. Dyotic signaling leads to suppressed immune function, promoting the development of neoplasia (Chahal and Drake 2007; Gameiro and Romao 2010; Gameiro, et al. 2010).
9. Diabetes mellitus. Dyotic signaling alters fuel metabolism leading to obesity, insulin insensitivity, metabolic syndrome and diabetes mellitus type II (Atwood and Bowen 2007; Clark, et al. 2012).
10. Rheumatoid arthritis. Elevated LH and FSH promote the onset and exacerbation of rheumatoid arthritis (Kass et al., 2010).

Before certain embodiments are described in greater detail, it is to be understood that this invention is not limited to certain embodiments described, and as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein have discrete components and features which may be readily separated from or combined with the features of any of the other several embodiments without departing from the scope or spirit of the present invention. Any recited method may be carried out in the order of events recited or in any other order which is logically possible.

### Methods of Treatment

The invention encompasses methods of treating male hypogonadism by implanting or injecting hormone-producing organoids (e.g., gonadal hormones).

The invention also encompasses a method of preventing or reversing the dysregulation of the HPG axis by injecting donor cells to repopulate the ovaries with follicular cells, and the testes with Leydig, Sertoli and other support cells, or injecting donor cells into extra-gonadal tissues. This will prevent and treat hypogonadism, prevent and treat symptoms associated with female reproductive endocrine dyscrasia and symptoms associated with male reproductive endocrine dyscrasia, and/or prevent and delay the onset of age-related diseases and extend longevity.

In certain embodiments, this repopulation is achieved by implanting functional gonadal organoids generated from progenitor cells that were differentiated into the gonadal organoids. In some embodiments, the organoids are generated from MSCs. In other embodiments, the gonadal organoids are generated from ADSCs. In other embodiments these gonadal organoids are injected into extra-gonadal tissues.

The invention encompasses a method of restoring the HPG axis to balance (young adult reproductive levels) by injecting donor cells to repopulate the ovaries with follicular cells, and the testes with Leydig, Sertoli and other support cells, or injecting donor cells into extragonadal tissue. This will reverse hypogonadism, prevent and treat symptoms associated with female reproductive endocrine dyscrasia and symptoms associated with male reproductive endocrine dyscrasia, and/or prevent and delay the onset of age-related diseases and extend longevity.

The invention further encompasses a method of inhibiting inflammation such as decreasing the expression of tumor necrosis factor (TNF), in a subject, by administering donor cells that lead to a rebalancing of the HPG axis.

Thus, the present invention encompasses reversing the degenerative serum hormonal milieu back to one that allows the appropriate growth and development of cells for the normal maintenance of tissue structure and function in the body. Rebalancing the endocrine HPG axis will allow for the rebalancing of the tissue specific ‘mini-HPG’ axes present in tissues throughout the body (Meethal et al. 2009b). This will rebalance reproductive hormone signaling to cells in all tissues of the body.

This can be achieved by injecting into a subject donor cells that can 1) repopulate the gonads, or 2) populate extra-gonadal tissue, with cell types capable of producing reproductive hormones required to balance the HPG axis. For male subject, donor cells capable of differentiating into germ cells (spermatogonia, spermatocytes, spermatids, and spermatozoon), Sertoli cells, myoid cells, Leydig cells, stromal cells, macrophage cells and/or epithelial cells and integrating into the tissue to restore function. For female subject, donor cells capable of differentiating into germ cells (oogonial stem cells), granulosa cells, cumulus cells, thecal cells, stromal cells, epithelial cells, macrophage cells and/or oocyte cells, and integrating into the tissue to restore function.

The differentiation of donor cells into more than one gonadal cell type is required to allow complete rebalancing of the axis. For example, while Leydig cells primarily produce androgens, Sertoli cells produce large quantities of inhibins and AMH, both of which are required for HPG axis rebalancing in males.

An embodiment of the present invention includes administering, to a subject, donor cells that decrease or regulate the blood levels, production, function, or activity of gonadal hormone to be near the blood levels, production, function or activity occurring during fetal life or at or around the height of the subject's reproductive period, which in humans usually corresponds to about 18 to 35 years of age.

In another embodiment, the present invention encompasses administering, to a subject, donor cells that decrease or regulate the blood levels, production, function or activity of kisspeptin, GnRH, LH or FSH to be approximately as low as possible without significant adverse side effects, preferably to be undetectable or nearly undetectable by conventional detection techniques known in the art, which, at the present time, is less than 0.7 mIU/mL for both LH and FSH. In another embodiment, the present invention encompasses administering, to a subject, donor cells that regulate the function or activity of activin to be approximately as low as possible without significant adverse side effects, preferably to be undetectable or nearly undetectable by conventional detection techniques known in the art. In another embodiment, the present invention encompasses administering donor cells that increase or regulate the blood levels, production, function, or activity of inhibin, follistatin, myostatin or BMP4 to be approximately as high as possible without significant adverse side effects.

In other embodiments of the present invention, the blood levels, production, function or activity of gonadal hormones are continuously regulated, by monitoring the blood levels, production, function or activity and making adjustments to the donor cell or donor cells being administered via a feedback control system.

In certain embodiments, the donor cells are donor progenitor or stem cells, such as mesenchymal stem cells, that have been differentiated in vitro into hormone producing organoids.

Embodiments of the present invention include administration of one or more stem or differentiated cell types that can be used to increase or regulate the blood and/or tissue levels, production, function or activity of gonadal hormones. In certain embodiments, the methods include administration of the gonadal organoids. Studies have shown that increasing the levels of circulating sex steroids and inhibins will result in significant decreases in GnRH, LH and FSH levels and a rebalancing of the HPG axis (Hayes, et al. 1998; Thorner et al. 1998; Ying 1988). Through a negative feedback loop, the presence of sex steroid hormones such as estrogen, testosterone or progesterone signals the hypothalamus to decrease the secretion of GnRH (Gharib, et al. 1990; Steiner, et al. 1982). The subsequent decrease in GnRH decreases the secretion of LH and FSH (Thorner et al. 1998). For example, sex steroids, inhibins and follistatin have been shown to provide negative feedback regulation of GnRH and FSH synthesis and secretion (Bagatell, et al. 1994; Boepple, et al. 2008; Dubey, et al. 1987; Hayes, et al. 2001b; Illingworth, et al. 1996; Lambert-Messerlian, et al. 1994; Marynick, et al. 1979; Pitteloud, et al. 2008a, b; Schnorr, et al. 2001; Sherins and Loriaux 1973; Winters, et al. 1979a; Winters, et al. 1979b) while sex steroids and gonadotropin surge-attenuating factor (GnSAF) appear to primarily provide negative feedback for the regulation of GnRH and LH synthesis and secretion (Bagatell et al. 1994; Hayes, et al. 2001a; Santen 1975; Schnorr et al. 2001; Veldhuis, et al. 1992; Messinis et al., 2018). In females, sex steroids, inhibins and follistatin have been shown to provide negative feedback regulation of FSH (le Nestour, et al. 1993; Welt, et al. 1997) and LH (Jaffe and Keye 1974, 1975; Jaffe, et al. 1976; Keye and Jaffe 1974, 1975, 1976; Liu and Yen 1983; Taylor, et al. 1995; Young and Jaffe 1976) synthesis and secretion.

Embodiments of the present invention also encompass rebalancing of the HPG axis such that the axis and related hormonal concentrations are balanced for that person. The production of sex hormones by donor cells is expected to be different for different individuals in order to reach optimal balancing of that person's HPG axis. Thus, the circulating and tissue concentrations of sex hormones in one person's balanced HPG axis is expected to be different to that of another person whose axis is also balanced.

Embodiments of the present invention also encompass the minute-to-minute, hour-to-hour and day-to-day variations in HPG axis hormone production to allow the axis to remain in balance.

Embodiments of the present invention encompass a method for treating male hypogonadism in a subject in need of treatment includes administering testicular organoids generated from isolated male progenitor cells. In certain embodiments, the method restores hormone balance in the subject. In some embodiments, the subject is not concurrently receiving testosterone replacement therapy. In certain embodiments, the hypogonadism being treated is testosterone deficiency syndrome (TDS) or congenital or acquired primary hypogonadism. In other embodiments, the subject is receiving testosterone replacement therapy or other therapies that partially rebalance the HPG axis. In certain preferred embodiments, the subject does not receive pre-treatment with chorionic gonadotrophin (CG) prior to being administered the testicular organoids.

Further embodiments of present invention encompass a method of increasing healthspan or longevity in a male subject in need thereof comprising administering to the subject testicular organoids generated from isolated male progenitor cells. In embodiments, the subject is not concurrently receiving testosterone replacement therapy. In preferred embodiments, the subject does not receive pre-treatment with chorionic gonadotrophin (CG) prior to being administered the testicular organoids.

Additional embodiments of the invention encompass a method of rebalancing the HPG axis in a male subject in need thereof comprising administering to the subject testicular organoids generated from isolated male progenitor cells. In some embodiments, the subject is not concurrently receiving testosterone replacement therapy. In certain preferred embodiments, the subject does not receive pre-treatment with chorionic gonadotrophin (CG) prior to being administered the testicular organoids.

In some embodiments, the subject receiving treatment is a male mammal. Thus, the subject being treatment may be a male human, such as for example a human 50 years of age or older.

The testicular organoids can be administered by a variety of routes. In one embodiment, the method includes injecting the testicular organoids, such as for example, subcutaneous injection.

A variety of different progenitor cell sources can be used in the methods. In certain embodiments, the male progenitor cells used to generate the testicular organoids are isolated from the subject being treated. In other embodiments, the male progenitor cells used to generate the testicular organoids are isolated from commercial cell line. In some embodiments, the progenitor cells are adult-derived mesenchymal stem cells, embryonic-derived stem cells, umbilical-cord-derived mesenchymal stem cells, or adipose-derived stem cells (ADSCs).

While the precise make up of a testicular organoid may vary, in some embodiments, the testicular organoid includes cells expressing GFRA1, VEGF, VEGFR2, PDGF-Rα, CYP11A1, 3β-HSD, 17β-HSD, LHCGR, α-SMA, CD11b, MHC class II, CD64, CD95, Sox9, AR, inhibin β-B, and FSHR.

The method can include further steps. For example, in some embodiments, the method further includes pre-treating the subject with chorionic gonadotrophin (CG). In other preferred embodiments, the method excludes pre-treating the subject with chorionic gonadotrophin (CG). In certain embodiments, generating the testicular organoids includes expanding isolated male progenitor cells, generating a spheroid under dynamic culture conditions, and culturing that spheroid under dynamic culture conditions to produce a testicular organoid. Alternatively or additionally, in other embodiments, culturing the spheroid under dynamic conditions includes using a culture medium supplemented with luteinizing hormone (LH), follicle-stimulating hormone (FSH), a thyroid hormone, human insulin-like growth factor-1 (IGF-1), human glial cell line-derived neurotrophic factor (GDNF), retinoic acid, smoothened agonist (SAG), 8-bromoadenosine-3′,5′-cyclic AMP (8-Br-cAMP), testosterone, and platelet-derived growth factor AA or AB to generate the testicular organoid.

Embodiments of the present invention also encompass returning the ratios of sex hormones back to near the ratios occurring during fetal life or at or near the time of greatest reproductive function of the subject. For example, the ratio of testosterone:FSH during the male reproductive period is ˜11 (6.5 ng/mL:0.6 ng/mL), while that during the post-reproductive period (andropause) is ˜1 (2.3 ng/mL:2.3 ng/mL). In females, the ratio of 17β-estradiol:FSH decreases from ˜0.015 during the reproductive period to ˜0.0004 during the post-reproductive period. In these examples, treatment would aim to return the ratio of these hormones back to ˜11 and 0.015, respectively. Further embodiments to this invention would encompass returning all the sex hormone ratios back to those during fetal life or at the time of greatest reproductive function of the subject.

Embodiments of the present invention also encompass administration of purified and mixed donor cell populations derived from the tissues of an individual who will receive the donor cells.

Embodiments of the present invention also encompass administration to an individual purified and mixed donor cell population derived from multiple tissues of one or more individuals.

Embodiments of the present invention encompass administration of autologous or allogenic donor cell populations into the gonads, or extra-gonadal tissue for the prevention or treatment of hypogonadism, hypergonadotropic hypogonadism, andropause, menopause and related conditions, and for the prevention and treatment of diseases associated with senescence and aging.

Embodiments of the present invention also encompass administration of donor cell populations into the gonads or extra-gonadal tissues to restore hormone balance concurrent with or prior to administration of donor cell populations (e.g. stem cell therapy, iPSC therapy, or implantation/injection of differentiated cells including stem cells that have been differentiated in vitro) into other tissues of the body. Such a method allows for rebalancing the HPG axis so that the ‘toxic environment of dyotic signaling’ is reversed in order to allow for donor cells transplanted into other tissues to differentiate appropriately, integrate into the tissue and restore function.

In other embodiments of the invention, donor cell recipients may receive supplemental gonadal hormones, GnRH agonists/antagonists, an LH/FSH-inhibiting agent, an activin-inhibiting agent, myostatin-inhibiting agent, an inhibin-promoting agent, and/or a follistatin-promoting agent.

According to embodiments of the present invention, administration of GnRH agonists/antagonists, LH/FSH-inhibiting agents, activin-inhibiting-agents, myostatin-inhibiting agent, inhibin-promoting agents, follistatin-promoting agents, or sex steroids, including those listed above, can be oral or by injection, inhalation, transdermal (e.g. patch or gel), or other effective means. According to embodiments of the present invention, the dosage of GnRH agonists/antagonists, LH/FSH-inhibiting agents, activin-inhibiting agents, myostatin-inhibiting agent, inhibin-promoting agents, follistatin-promoting agents, or sex steroids, including those identified above, will be a therapeutically effective amount, sufficient to decrease or regulate the blood and/or tissue levels, production, function or activity of GnRH, LH or FSH, or to decrease or regulate the function or activity of activin or to increase or regulate the blood and/or tissue levels, production, function or activity of inhibin or follistatin, to the desired blood and/or tissue levels, production, function or activity. According to other embodiments of the invention, administration of LH/FSH-inhibiting agents, activin-inhibiting agents, myostatin-inhibiting agent, inhibin-promoting agents, follistatin-promoting agents, or sex steroids, including those identified above, can be in a single dose, multiple doses, in a sustained release dosage form, in a pulsatile form, or in any other appropriate dosage form or amount. Administration prior to treatment with cells is preferred but can occur during or after administration of cells. The duration of treatment could range from a few days or weeks to the remainder of the patient's life.

In addition to treating neurodegenerative diseases, the administration of GnRH agonists/antagonists, LH/FSH-inhibiting agents, activin-inhibiting agents, myostatin-inhibiting agent, inhibin-promoting agents, follistatin-promoting agents, sex steroids, or other agents that decrease dysregulated cell cycle signaling, as described above, is expected to be beneficial in the treatment of aging and diseases where cell replenishment is required in order to repopulate a tissue to regain function or establish a new function, in accordance with the present invention.

### Methods of Generating Testicular and Ovarian Organoids

In certain embodiment, the disclosure provides for generation of testicular or ovarian organoids.

Generation of the testicular or ovarian organoids is achieved by culturing the progenitor (stem) cells, such as male or female MSCs in a medium supplemented with LH, FSH, a thyroid hormone, human insulin-like growth factor 1 (IGF-1), human glial cell line-derived neurotrophic factor (GDNF), retinoic acid, Smoothened agonist (SAG), 8-bromoadenosine 3′,5′-cyclic adenosine monophosphate, testosterone, and platelet-derived growth factor AA or platelet-derived growth factor AB.

In certain embodiments, the progenitor (stem) cells, are mesenchymal stem cells. In other embodiments, the stem cells are embryonic stem cells or induced pluripotent stem cells.

In certain embodiments, the culture medium comprises, i.e. is supplemented with, about 5 ng/ml of LH, about 10 ng/ml of FSH, about 1 nM of thyroid hormone, about 70 ng/ml of IGF-1, about 40 ng/ml of GNDF, about 200 ng/ml of retinoic acid, about 0.2 μM of SAG, about 0.1 mM of 8-Br-cAMP, about 10 nM of testosterone, and about 20 ng/ml of PDGF-AA or about 20 ng/ml of PDGF-AB.

In other embodiments, the culture medium comprises, i.e. is supplemented with, from about 1 ng/ml to about 10 ng/ml of LH, from about 2 ng/ml to about 20 ng/ml of FSH, from about 0.5 nM to about 1.5 nM of thyroid hormone, from about 10 ng/ml to about 150 ng/ml of IGF-1, from about 10 ng/ml to about 100 ng/ml of GNDF, from about 50 ng/ml to about 500 ng/ml of retinoic acid, from about 0.1 μM to about 1 μM of SAG, from about 0.025 mM to about 1 mM of 8-Br-cAMP, from about 1 nM to about 15 nM of testosterone, and from about 5 ng/ml to 50 ng/ml of PDGF-AA or from about 5 ng/ml to 50 ng/ml of PDGF-AB.

In another embodiment, the culture medium is supplemented with the above factors in a staged manner.

In certain embodiments, the thyroid hormone is triiodothyronine, thyroxine, an analogue of triiodothyronine, an analogue of thyroxine or a mixture thereof.

In certain embodiments, the culture medium is further supplemented with insulin-transferrin-selenium (ITS) and 22R-hydroxycholesterol. The culturing of the cell is carried out under conditions sufficient to initiate gonadal organoid formation. Alternatively, the culturing is carried under conditions sufficient to generate gonadal organoids. In certain embodiments, the culturing comprises culturing the cells for about 4-28 days, alternatively about 4 days, alternatively about 5 days, alternatively about 6 days, alternatively about 7 days, alternatively about 9 days, alternatively about 11 days, alternatively about 14 days, alternatively about 15 days, alternatively about 17 days, alternatively about 19 days, alternatively about 21 days, alternatively about 23 days, alternatively about 25 days, alternatively about 27 days, alternatively about 29 days. In certain embodiments, the culture medium is supplemented with serum albumin. In other embodiments, the culture medium lacks further differentiation agents.

The gonadal organoids comprise cell expressing markers of spermatogonial stem cells (GFRA1), vascular cells (VEGF, VEGFR2), Leydig cells (PDGF-Rα, Cyp11A1, 3β-HSD, 17β-HSD, LHCGR), myoid cells (α-SMA and CD11b), macrophages (MHC CII and CD64) and Sertoli cells (CD95, Sox9, AR, inhibin β-B, and FSHR). In certain embodiments, the gonadal organoids comprise about 10-25% of Leydig cell markers; about 50% of myoid cell markers; about 45% of macrophage markers; and about 5-30% of Sertoli cell makers (except for CD95).

In some embodiments, the gonadal organoids comprise cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR. In other embodiments the expression of cell markers varies throughout the time of differentiation. In other embodiments, the gonadal organoid comprises at least 10-25% cells expressing PDGF-Rα, Cyp11A1, 3β-HSD, 17β-HSD, and LHCGR, about 5-50% of cells expressing α-SMA and CD11b, about 5-50% of cells expressing MHC CII and CD64 and at least 5-30% of cells expressing Sox9, AR, inhibin β-B, and FSHR. In other embodiments, the gonadal organoids comprise at least 5-30% cells expressing PDGF-Rα, Cyp11A1, 3β-HSD, 17β-HSD, and LHCGR, about 5-15% of cells expressing α-SMA and CD11b, about 5-15% of cells expressing MHC CII and CD64 and at least 10-50% of cells expressing Sox9, AR, inhibin β-B, and FSHR, about 5-30% of cells expressing VEGF and VEGFR2, and cells expressing about 3-15% GFRA1. In other embodiments, the gonadal organoids comprise at least about 5, 10, 15, 20, 25, 27, or 30% cells expressing PDGF-Rα, Cyp11A1, 3β-HSD, 17β-HSD, and LHCGR, at least about 5, 6, 7, 8, 9, 10, 11, 12, 13, 14, or 15% of cells expressing α-SMA and CD11b, at least about, 6, 7, 8, 9, 10, 11, 12, 13, 14, or 15% of cells expressing MHC CII and CD64 and at least about 10, 15, 20, 25, 30, 35, 40, 45, or 50% of cells expressing Sox9, AR, inhibin β-B, and FSHR, at least about 5, 10, 15, 20, 25, or 30% of cells expressing VEGF and VEGFR2, and cells expressing at least about 3, 4, 5, 6, 7, 8, 9, 10, 11, 12, 13, 14, or 15% GFRA1. In alternate embodiments, the gonadal organoids comprise about 5, 10, 15, 20, 25, 27, or 30% cells expressing PDGF-Rα, Cyp11A1, 3β-HSD, 17β-HSD, and LHCGR, about 5, 6, 7, 8, 9, 10, 11, 12, 13, 14, or 15% of cells expressing α-SMA and CD11b about, 6, 7, 8, 9, 10, 11, 12, 13, 14, or 15% of cells expressing MHC CII and CD64 and about 10, 15, 20, 25, 30, 35, 40, 45, or 50% of cells expressing Sox9, AR, inhibin β-B, and FSHR about 5, 10, 15, 20, 25, or 30% of cells expressing VEGF and VEGFR2, and cells expressing about 3, 4, 5, 6, 7, 8, 9, 10, 11, 12, 13, 14, or 15% GFRA1. In certain embodiments, the disclosure also encompasses the gonadal organoids in the differentiation medium.

In other embodiments, the methods of generating the gonadal organoids further comprises spheroid formation from the progenitor cells prior to gonadal organoid formation. In other embodiments, the methods also including isolating the stem (progenitor) cells.

In certain embodiments, the methods of generating gonadal organoids combine static culture conditions with dynamic culture conditions, such as a spinner flask or bioreactor. In embodiments, the methods of generating the gonadal organoids require differentiating the progenitor cells into the hormone-producing organoids under dynamic conditions.

The methods further include expanding the cells prior to differentiation in an expansion medium containing a rho-kinase inhibitor such as H1142.

In some embodiments, the method further includes formulating the gonadal organoid for implantation. In instance, the gonadal organoids may be formulated as a part of a matrix.

In other embodiments, the gonadal organoids can be incorporated into a three-dimensional support prior to implantation. The gonadal organoids can be maintained in vitro on this support prior to implantation into the patient. Alternatively, the support containing the gonadal organoids can be directly implanted in the patient without additional in vitro culturing. The support can optionally be incorporated with at least one pharmaceutical agent that facilitates the survival and function of the transplanted gonadal organoids.

Embodiment of the present invention encompass a method for generating a testicular organoid includes producing a spheroid from isolated male progenitor cells under dynamic conditions, and then culturing the spheroid under dynamic conditions in a medium supplemented with LH, FSH, a thyroid hormone, IGF-1, GDNF, retinoic acid, SAG, 8-Br-cAMP, testosterone, and platelet-derived growth factor AA or AB to form the organoid.

In some embodiments of the method, the testicular organoid includes cells expressing GFRA1, VEGF, VEGFR2, PDGF-Rα, CYP11A1, 3β-HSD, 17β-HSD, LHCGR, α-SMA, CD11b, MHC class II, CD64, CD95, Sox9, AR, inhibin β-B, and FSHR. In other embodiments, the testicular organoid includes at least 5-30% of cells expressing PDGF-Rα, CYP11A1, 3β-HSD, 17β-HSD, and LHCGR; about 5-50% of cells expressing α-SMA and CD11b; about 5-50% of cells expressing MHC class II and CD64; at least 5-30% of cells expressing Sox9, AR, inhibin β-B, and FSHR; about 5-30% of cells expressing VEGF and VEGFR2; and about 3-15% of cells expressing GFRA1.

In some embodiments, the progenitor cells used in the method are adult-derived mesenchymal stem cells, embryonic stem cells, umbilical-cord-derived mesenchymal stem cells, or adipose-derived stem cells.

The duration of culturing can vary. In some embodiments of the method, the cells are cultured for at least 7 days or for approximately 14 to 27 days.

The method can include further steps. For example, in some embodiments, the method further includes culturing the progenitor cells in a medium supplemented with LH, FSH, a thyroid hormone, IGF-1, GDNF, retinoic acid, SAG, 8-Br-cAMP, testosterone, and platelet-derived growth factor AA.

The following examples are offered by way of illustration and not by way of limitation.

### EXAMPLES

### Example 1

Adult stem mesenchymal stem cells (MSCs) (or bone marrow stromal cells) are pluripotent cells that have the ability to differentiate into cells of all three germ layers (Ratajczak, et al. 2008). MSCs injected into the testes localize to the testicular interstitium and seminiferous tubules and differentiate into Leydig cells and spermatogonia/spermatocytes, respectively (Lo, et al. 2004; Yazawa, et al. 2006). Stem cells injected into the ovaries increase follicle numbers (Abd-Allah, et al. 2013). Hormonal factors secreted within the gonads direct the differentiation and integration of such stem cells for the replenishment of germ cells, Leydig, Sertoli and other cells in the testes, and replenishment of follicular cells (germ cells, granulosa, thecal and other cells) in the ovaries. Hormones secreted by the transplanted cells and their progeny in turn rebalance the HPG axis.

In the case of a human male or female, MSCs are isolated from 1) bone, the femur and/or tibia (Tuli, et al. 2003a; Tuli, et al. 2003b), 2) umbilical cord blood (Hayward, et al. 2013; Malgieri, et al. 2010), 3) Wharton's jelly (Hayward et al. 2013), 4) skin (Manini, et al. 2011) or 5) adipose tissue (Kuhbier, et al. 2010; Manini et al. 2011; Tholpady, et al. 2003; Zhu, et al. 2013; Zuk, et al. 2001). Cells are then subjected to flow cytometry to isolate MSC that are then injected (10,000-1 billion cells/treatment) into the interstitium of one or both testes or ovaries of the donor. In the testes, cells can be injected into the seminal vesicle lobules, septa, tunica albuginea, straight tubule, rete testes, efferent ductile and/or epididymis. In the ovary, cells can be injected into the ovarian cortex. If the number of isolated MSCs is insufficient, MSCs are expanded in culture first prior to injection into the gonads.

This technique may be performed autologously, i.e. isolating cells from the same individual who will receive the cells; allogeneically, i.e. cells isolated from one individual are injected into another individual (human or animal); or both autologously and allogeneically, i.e. isolated cells from the recipient and from another individual(s) are injected into the recipient.

In this example, MCSs, from which gonadal tissues are derived during embryogenesis, are purified from tissues other than the gonads and then injected into the gonads. MSCs in a suitable buffer or encapsulated in a hydrogel or other matrix (e.g. fibrin, collagen) prior to injection, may be injected into the gonads (testes or ovaries). Injection may be via a catheter. MSCs in this example are capable of differentiating into all relevant gonadal cell types upon injection into the gonads.

This technique can be used on humans, animals and plants with a reproductive hormone axis.

The concentration of circulating reproductive hormones in the individual can be measured before and after the injection of cells to confirm that injected cells are producing hormones and rebalancing the HPG axis. Tissue concentrations of reproductive hormones can be measured in tissues to confirm that the hormones of the mini-HPG-axis in that tissue have rebalanced (returned to young adult reproductive concentrations). If the HPG axis has not completely rebalanced, a second or subsequent injection can be given until such time as the HPG axis is balanced and dyotic signaling has decreased. This provides a preventative and treatment for hypogonadism (primary) and of age-related reproductive endocrine dyscrasia.

### Example 2

MSCs or other stem cell populations are differentiated in vitro into discrete precursor or differentiated cell types including germ cells (spermatogonia, spermatocytes, spermatids and spermatozoon), Sertoli cells, myoid cells, Leydig cells, stromal cells, macrophage cells, endothelial cells and/or epithelial cells in the case of the male; or germ cells (oogonial stem cells), granulosa cells, cumulus cells, thecal cells, stromal cells, epithelial cells, endothelial cells, macrophage cells and/or oocyte cells, in the case of the female, and one or preferably more of these cell types are injected into the gonads and/or other tissues and circulating and tissue sex hormone concentrations measured as in the methods described in Example 1, for rebalancing of the HPG axis.

### Example 3

Adult testicular cells such as Sertoli cells, Leydig cells and germ cells can be differentiated from MSCs following transfection with members of the nuclear receptor family, SF1 or liver receptor homolog-1 (LRH-1), and treatment with 8-bromoadenosine-cAMP (Yazawa et al. 2006). One or preferably both of these cell types are injected into the male gonads and/or other tissues neat or in matrices via methods described in Example 1 and circulating and tissue sex hormone concentrations measured as in the methods described in Example 1, for rebalancing of the HPG axis. Cells may be autologous or allogeneic. In a derivation of this method, MSC or other cell types are treated with differentiation factors as described in Example 3 and injected within 24 h into the gonads and/or other tissues via methods described in Example 1. In another derivation of this method, MSC or other cell types are imbedded in a matrix impregnated with differentiation factors and injected into the and/or other tissues via methods described in Example 1.

Adult granulosa, cumulus, thecal and germ cells can be isolated from adult ovaries following trituration, percoll gradients and/or flow cytometry (Sittadjody, et al. 2013) and one or preferably more of these cell types injected into the female gonads and/or other tissues and circulating and tissue sex hormone concentrations measured as in the methods described in Examples 1 and 3, for rebalancing of the HPG axis.

### Example 4

Donor cells derived from the gonads of the recipient are differentiated into discrete precursor or differentiated cell types including germ cells (spermatogonia, spermatocytes, spermatids and spermatozoon), Sertoli cells, myoid cells, Leydig cells, stromal cells, macrophage cells, endothelial cells and/or epithelial cells in the case of the male; or germ cells (oogonial stem cells), granulosa cells, cumulus cells, thecal cells, stromal cells, epithelial cells, endothelial cells, macrophage cells and/or oocyte cells, in the case of the female, and one or preferably more of these cell types are injected into the gonads and/or other tissues and circulating and tissue sex hormone concentrations measured as in the methods described in Example 1, for rebalancing of the HGP axis.

### Example 5

Donor cells derived from the gonads of the recipient are treated with demethylation agents to allow for epigenetic silencing. As in the methods described in Example 1, these cells are injected into the gonads and/or other tissues and circulating and tissue sex hormone concentrations measured.

### Example 6

Cells derived from somatic-cell nuclear transfer (SCNT) into an enucleated oocyte (Byrne, et al. 2007) can be cultured to produce sufficient cell numbers to be injected into either one or both of the gonads, and/or injected into the circulation, and/or other tissues of the body and circulating and tissue sex hormone concentrations measured as described in Example 1 to rebalance the HPG axis. The nucleus used for SCNT may be from the same individual who is receiving the injection, or from a different individual than who is receiving the injection. The enucleated oocyte may be from the same individual who is receiving the injection, or from a different individual than who is receiving the injection.

### Example 7: This Technique Can be Used on Humans, Animals, and Plants With a Reproductive Hormone Axis

Induced pluripotent stem (iPS) cells created from the recipient or another donor can be cultured to produce sufficient cell numbers to be injected into either one or both of the gonads, and/or injected into the circulation, and/or other tissues of the body and circulating and tissue sex hormone concentrations measured as described in Example 1 to rebalance the HPG axis. Differentiated cells such as fibroblasts, umbilical cord fibroblasts stomach, hepatocytes, lymphocytes, prostatic cells and other adult differentiated cells can be obtained by various techniques known in the field and reprogrammed into iPS cells via the following techniques also known in the field.

Generation of iPSCs Reprogramming With Lentiviral Transduction

Three plasmid vectors of lentiviral reprogramming: FUW-tetO-lox-hO2S, FUW-tetO-lox-hM2K, and FUW-tetO-lox-hN2L are constructed. Expression cassettes of human POU5F1-internal ribosome entry site 2 (IRES2)-SOX2 (O2S) and MYC-IRES2-KLF4 (M2K) of pEP4 EO2S EM2K (Addgene, #20923) (Yu, et al. 2009) are used for the O2S and M2K cassettes. Pseudovirus is produced in 293FT cells by transfection with each lentiviral vector (O2S, M2K, N2L) and the reverse tetracycline transactivator expression plasmid, FUW-M2rtTA (Addgene, plasmid 20342) (Hockemeyer, et al. 2008) along with the VSV-G envelope (pMD2.G) and packaging vector (psPAX2) (Ezashi, et al. 2009). Two consecutive infections are introduced into the target cell or interest (1×10⁵ cells) in the presence of 12 μg/ml hexadimethrine bromide (polybrene, Sigma, St. Louis, MO). During the infection stage, the cells are cultured for 48 h by adding a mixture of the four titered pseudoviruses (multiplicity of infection); O2S (30.8), M2K (17.5), N2L (18.2) and rtTA (20.7) to the culture medium. On day 4 after infection, cells are dispersed with trypsin and then expanded. Cells are tested for pluripotency and can then be used for treatment.

### Reprogramming With Episomal Plasmids

Episomal vectors carrying the reprogramming genes SOX2, KLF4, POU5F1, LIN28, p53 and MYCL (combined episomal plasmids; Addgene #27077, 27078 and 27080) are electroporated into 1-6×10⁵ cells using a Nucleofector II device (Lonza, Basel, Switzerland) and Amaxa NHDF Nucleofector kit (Lonza). After 20 days, colonies resembling human ESC are mechanically isolated and expanded in mTeSR1 medium (Gallego et al. 2010; Ludwig, et al. 2006; Porayette, et al. 2009) (StemCell Technologies, Vancouver, Canada) on a Matrigel (BD Bioscience, San Jose, CA) coated substratum. Cells are tested for pluripotency and can then be used for treatment.

### Example 8: Subcutaneous Injection of Mesenchymal Stem Cells Differentiated into ‘Testicular’ Organoids Reverses Hypogonadism in a Castrated Rodent Model

### Methods

### Preparation of Functional Organoids for Transplantation Studies of Efficacy and Safety

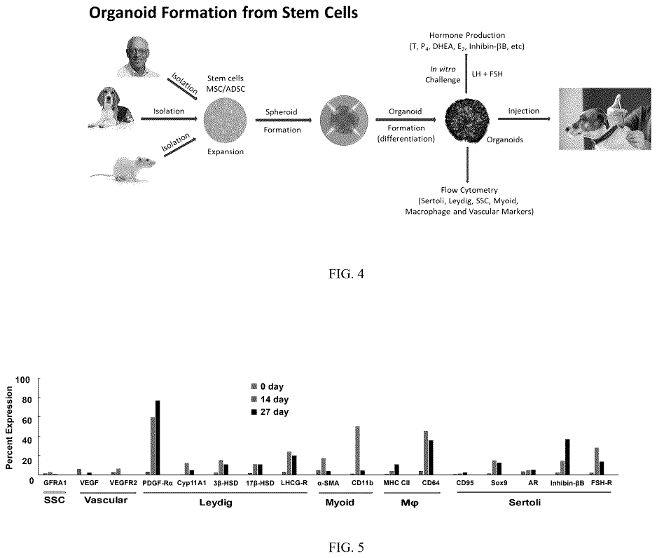

The generation of functional gonadal organoids that can be injected into non-gonadal sites provides a base technology for the restoration of hormone balance in both intact and oophorectomized/orchiectomized individuals. Based on the transcription factors required to generate macrophage, Leydig, myoid, Sertoli and SSC's (i.e., SF1, WT1, GATA-1 GATA-4, DMRT1 and cMYC), we screened over 200 combinations of developmental hormones and growth factors known to mediate their actions via these transcription factors for the development of a media that could drive stem cells into sex hormone-producing organoids. We have formulated a media (Cellular Differentiation Media-3.4; CDM-3), composed of a combination of developmental hormones and growth factors, which allows the differentiation of neonatal rat bone-marrow derived mesenchymal stem cells (rBM-MSC), rat adipose-derived stem cells (rADSC), neonatal canine bone marrow-derived MSCs (cBM-MSC) and canine adipose-derived stem cells (cADSC) into gonadal organoids complete with six crucial cell types that support optimal hormone production. This media also allows the differentiation of vascular endothelial cells and the potent angiogenesis factor, vascular endothelial growth factor (VEGF), in these gonadal organoids to promote in vivo angiogenesis and anastomosis, important for the long-term perfusion of nutrients to these mini organs. A schematic diagram of the steps involved in the generation of testicular organoids, as an example, is provided in FIG. 4.

MSC Gonadal Organoid Production: rBM-MSC are isolated from the marrow of long bones of a neonatal Fischer rat. Using a 1 mL syringe and a 27-gauge needle, DMEM/F12 (no phenol red) media, is injected through the bone to flush out bone marrow that is then triturated with a pipette. Red blood cell lysis buffer (1 mL; Invitrogen™, eBioscience™, Santa Clara, CA) is mixed with the bone-marrow solution for 5 min. at 4° C., lysis stopped by adding Quench media (90% DMEM/F12+10% FBS) and the mixture centrifuged for 5 min. at 350 g. The supernatant is removed, and the bone marrow pellet resuspended in DMEM/F12 (no phenol red) media, recentrifuged and supernatant removed prior to resuspending the pellet and plating in Expansion Media consisting of DMEM/F12 (JangoCell, Fitchburg, WI) with 5-20% FBS, 1× Glutamax (ThermoFisher Scientific, Waltham, MA), and penicillin/streptomycin/amphotericin (PSA; ThermoFisher Scientific, Waltham, MA) in C300 cell culture flasks (TPP, Switzerland) in a cell culture incubator at 37° C. with 5% CO₂. After reaching 80-90% confluence, rBM-MSC is passaged every 5-7 days. Once the required number of cells have been generated, they are washed in phosphate-buffered saline (PBS; ThermoFisher Scientific, Waltham, MA) and collected by treating with 0.25% Trypsin-EDTA (ThermoFisher Scientific, Waltham, MA) for 5 min. followed by neutralization with Expansion Media. The cell solution is then centrifuged at 350 g for 5 min., the supernatant removed, and the cell pellet resuspended in Spheroid Media (100 mL-100 L Expansion Media plus rho-kinase inhibitor (H1152; 0.1 mL-100 mL; AdipoGen Corporation, San Diego, CA)) and transferred to a spinner flask/bioreactor (100 mL-100 L) maintained at revolutions per minute required to keep cells/aggregates/spheroids suspended in media and maintained at 37° C. with 5% CO₂. Spheroid Media is replaced with Expansion Media after 1-2 days to allow the formation of spheroids of ˜20-400 μM diameter (optimally 50-200 μM) and the media then exchanged at spinner flask day 5 with Complete Differentiation Media 3.4 (CDM-3.4: DMEM/F12, HEPES, no phenol red (ThermoFisher Scientific, Waltham, MA), 1% bovine serum albumin (Sigma, St. Louis, MO), 1× PSA (ThermoFisher Scientific, Waltham, MA), 1× Glutamax (ThermoFisher Scientific, Waltham, MA), 1× insulin-transferrin-selenium (ITS) solution (ThermoFisher Scientific, Waltham, MA), luteinizing hormone (LH, 5 ng/mL; Sigma, St. Louis, MO), follicle-stimulating hormone (FSH, 10 ng/mL; Sigma, St. Louis, MO), thyroid hormone (1 nM; Tocris, Minneapolis, MN), putrescine (100 μM; MP Biomedicals, Santa Ana, CA), human insulin-like growth factor 1 (IGF-1, 70 ng/mL; Shenandoah, Warminster, PA), human glial cell line-derived neurotrophic factor (GDNF, 40 ng/mL; Shenandoah, Warminster, PA), retinoic acid (200 ng/mL; Cayman Chemical, Ann Arbor, Michigan), smoothened agonist (SAG, 0.2 μM; Cayman Chemical, Ann Arbor, Michigan), 22R-hydroxycholesterol (5 μM; Cayman Chemical, Ann Arbor, Michigan), 8-bromoadenosine 3′,5′-cyclic adenosine monophosphate (8-Br-cAMP, 0.1 mM; Sigma, St. Louis, MO), testosterone (10 nM; Sigma, St. Louis, MO) and platelet-derived growth factor AA (PDGF-AA, 20 ng/mL; Shenandoah, Warminster, PA), for 14 days or 27 days. With other species, PDGF-AA can be exchanged for PDGF-AB. On the day of rat injection, the gonadal organoids formed in CDM3.4 are allowed to settle in the spinner flasks and are then collected, washed in PBS, resuspended in DMEM/F12 with ITS, placed on ice and then injected subcutaneously into the back of the neck of Fisher rats using a 22 G, 1′ needle under aseptic conditions.

ADSC Gonadal Organoid Production: Similar methodology is used to generate rADSCs. Briefly, inguinal adipose tissue (1-2 grams) is resected from young male rats, placed in 5-10 mL of transport solution (UW Cold Storage Solution; Preservation Solution Inc., Elkhorn, WI) at 4° C. and the tissue washed three times with 20 mL 1× PBS, pH 7.4 (ThermoFisher Scientific, Waltham, MA) containing PSA. Tissue is then chopped into small pieces, vigorously mixed with enzymatic solution (1 g of tissue/25 mL PBS+P/S, containing collagenase (25 mg), hyaluronidase (3.75 mg) and DNase I (0.5 mg)) and then incubated at 37° C. for 2 h with mixing every 10 min. The enzymatic reaction is stopped by adding an equal volume of Quenching Solution (DMEM/F12 containing 10% FBS) and the mixture then serially strained through 100 μm followed by 40 μm strainers prior to the filtrate being centrifuged (5 min. at 500 g). The pellet is resuspended in Red Blood Cell Lysis Solution (5 mL; ThermoFisher Scientific) for 5 min. at 4° C. prior to adding 5 mL of Quenching Solution. The solution is then centrifuged at 500 g for 5 min., the supernatant aspirated using a glass pipette and the pellet resuspended in Expansion Media and cultured in a 6-well plate at 37° C. in 5% CO₂ for 24 h. After reaching 80-90% confluence, rADSC are passaged until sufficient cells are obtained to move into a spinner flask to commence Spheroid Formation followed by their differentiation in CDM3.4 as described above.

Flow cytometry characterization of gonadal organoids: Gonadal organoids generated as described above are monitored for changes in specific markers of differentiation. ˜200,000 cells are stained for each marker. The Attune NxT Flow Cytometer is used to collect >50,000 events for each marker and gated on live cells using a fixable Live/Dead cell stain kit. Positive populations are ascertained using fluorescence minus one (FMO) for each fluorochrome. Cell markers are listed below in order of differentiation stage—from least differentiated (i.e. stem cells, precursor cells) to almost fully differentiated, with antibody details in parentheses: Spermatogonial stem cell (SSC) marker: GFRA1 (MsIgG1, Santa Cruz Biotechnology); Vascular cell markers: VEGF (Rabbit Polyclonal, TFS) and VEGF receptor (Rabbit Polyclonal, TFS)—adult endothelial cell markers; Leydig cell markers: PDGF-Rα—stem Leydig cell (SLC) marker (MsIgG1 Biotin conjugated, TFS), Cyp11A1—SLC marker (Rabbit Polyclonal, Abcam), 3β-HSD (MsIgG3 PE conjugated, Santa Cruz Biotechnology), 3β-HSD—progenitor Leydig cell (PLC) marker (MsIgG1, Santa Cruz Biotechnology), 17β-HSD (MsIgG1, Santa Cruz Biotechnology) and luteinizing hormone/chorionic gonadotropin receptor (LHCGR; MsIgG1, Novus Biologicals)—adult Leydig cell (ALC) markers; Myoid Cell Markers: α-SMA (MsIgG2a, TFS) and CD11b (MsIgG1, TFS); Macrophage markers: MHC CII (MSIgG1, ThermoFisher Scientific (TFS)) and CD64 (MsIgG1 PerCP-eFluor 780 conjugated, TFS); Sertoli cell markers: CD95—stem Sertoli cell marker (MsIgG1 eFluor 450 conjugated, TFS), Sox9—precursor Sertoli cell marker (Rabbit polyclonal, LifeSpan BioSciences, Inc), androgen receptor (MsIgG1 Biotin conjugated, TFS), inhibin B-β (MsIgG2, Santa Cruz Biotechnology) and FSH-R—adult Sertoli cell markers (MsIgG1, Novus Biologicals).

Hormone characterization of gonadal organoids: Gonadal organoids are washed twice with DMEM/F12 media and then challenged with 5 ng/mL LH, 10 ng/mL FSH (both from National Hormone & Peptide Program, Harbor-UCLA Medical Center, Torrance, CA) and hCG (10 mIU/mL, Pregnyl, Merck & Co., Inc, NJ) in DMEM/F12 with ITS media every other day for 4 days. The media is then collected, the volume balanced and analyzed for sex steroids using electrochemiluminescence (Mesoscale Diagnostics, LLC). Steroids are normalized to cellular protein measured using the Lowry method as modified by Atwood et al., (1992).

Rat injection, blood collection and hormone analyses: Castrated Fischer rats (n=3/group; 6 total) are injected with hCG (50 IU; Chorulon, Merck & Co., Inc, NJ) 7 days before and 14 days after injection of gonadal organoids, on alternate days (−7, −5, −3, −1, 1, 3, 5, 7, 9, 11, 13, 15). This amount of hCG in rats translates to about 100 IU/kg of bodyweight. More generally, a dosage of 50-200 IU/kg or even 10-10,000 IU/kg may be suitable for injection every 2-4 days. At day 0, rats are injected subcutaneously with 1 mL of gonadal organoids (26.8 million cells/mL suspended in DMEM/F12+ITS) into the back of the neck. Blood is collected from the animals: pre-castration, post-castration, pre-injection (Day 0) and then at intervals up to 30-weeks post-injection to obtain plasma. Plasma sex hormone levels are assessed using electrochemiluminescence with a 4-Spot Custom Steroid Hormone Panel (progesterone (P4), dehydroepiandrosterone (DHEA), T, 17β-estradiol (E2)) on an MSD Technology Platform (Mesoscale Diagnostics LLC, Rockville, MD).

### Results

### Flow Cytometry

Characterization of 14- and 27-day CDM-3.4 differentiated gonadal organoids by flow cytometry indicated the presence of all prominent testicular cell types as demonstrated by well-characterized markers of spermatogonial stem cells (GFRA1), vascular cells (VEGF, VEGFR2), Leydig cells (PDGF-Rα, Cyp11A1, 3β-HSD, 17β-HSD, LHCGR), myoid cells (α-SMA and CD11b), macrophages (MHC CII and CD64) and Sertoli cells (CD95, Sox9, AR, inhibin B-β, FSHR) (FIG. 5).

FIG. 5: Treatment of rBM-MSCs with CDM-3.4 increases markers of all testicular cell types including spermatogonial (SSC), vascular (endothelial), Leydig, myoid, macrophage (Mø) and Sertoli cells. rBM-MSC's are isolated (0 day) and differentiated in CDM-3.4 for either 14 or 27 days as described above and monitored for changes in specific markers of differentiation by flow cytometry.

After 14-days of differentiation, Leydig cell markers increased from low baseline levels of 1-2% to ˜10-25%, myoid cell markers from ˜3% to ˜50%, macrophage markers from ˜3% to ˜45%, Sertoli cell markers from ˜2% to 5-30% (except for CD95), and vascular cell and SSC markers did not change appreciably. 27-day differentiation did not increase the expression of markers, and in the case of α-SMA, CD11b and FSH-R decreased expression as compared with 14-days of differentiation. Importantly, inhibin B expression indicated the capacity for gonadal organoids to negatively signal to the hypothalamus/pituitary in vivo, while the presence of LHCGR and FSHR indicates the capacity of the gonadal organoids to respond in vitro and in vivo to LH/CG and FSH signaling for steroidogenesis.

### Hormone Analyses

In vitro: T concentrations in the media of gonadal organoids (27-day differentiated) challenged with LH, FSH and hCG for 4 days increased 2.2-fold over untreated gonadal organoids, indicating that LH/FSH signaling induced steroidogenesis.

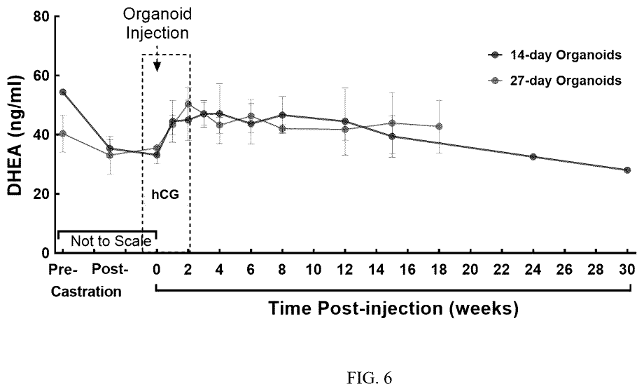

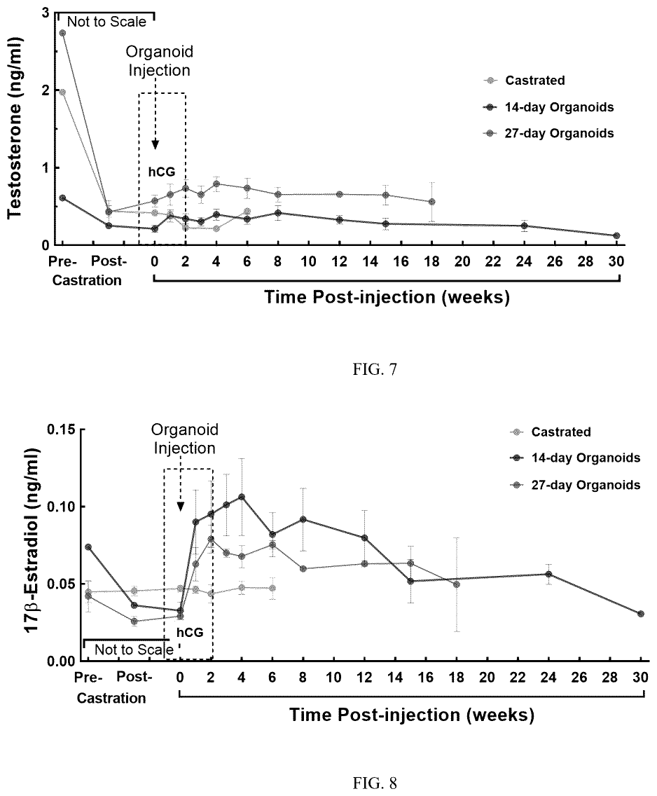

In vivo: Castration results in significant declines in plasma DHEA, T and E₂ concentrations in male Fisher rats (FIGS. 6-8, respectively). Injection of gonadal organoids differentiated for 14 days into rats injected with hCG (Chorulon, Intervet Inc., Merck Animal Health, NJ) prior to and following gonadal organoid injection increased DHEA, T and E₂ concentrations by 1-week, an effect that is maintained following the cessation of hCG injections at 2-weeks post-injection. Steroid hormones remained elevated for at least 12 weeks. Similarly, rats injected with gonadal organoids differentiated for 27 days plus hCG showed similar plasma DHEA and E₂ concentrations, but higher T concentrations, as compared to castrated rats injected with 14-day differentiated gonadal organoids plus hCG. In both groups of rats, steroidogenic hormones followed a similar concentration pattern within each animal (data not shown), supporting the functionality of the steroidogenic synthetic pathway within the injected gonadal organoids. hCG administration alone did not increase plasma T or E₂ concentrations, indicating that hCG alone 1) does not induce extra-testicular T or E₂ synthesis, and 2) that hCG induces sex hormone production from the injected gonadal organoids, likely by promoting their integration, proliferation, differentiation, and vascularization. In summary, CDM3.4-differentiated gonadal organoids from BM-MSCs injected into the neck (a non-testicular environment) of castrated rats increases circulating concentrations of DHEA and E₂ to around pre-castration concentrations, while elevations in T while significant, are lower than pre-castration concentrations. It remains to be determined if these lower circulating T concentrations are a new set point in these aging animals. These methods of gonadal organoid generation and their injection into animals together with hCG demonstrate the utility of this treatment for hypogonadism.

FIGS. 6-8: Injection of castrated hCG-treated Fisher rats with gonadal organoids increases circulating sex steroid concentrations. Fisher rats are injected subcutaneously into the back of the neck with testicular-like organoids (26.8 million cells; 14- or 27-day differentiated). One week prior to, and 2-4 weeks after injection, rats receive either saline or hCG (50 IU every other day). Plasma is collected pre-castration, post-castration, pre-injection of cells or saline injection, and then at intervals post-injection up to 30 weeks for analysis of sex steroids: DHEA, testosterone and E₂. The plasma hormone concentrations from animals injected with 14-day differentiated gonadal organoids+hCG (blue line; mean±SEM; n=3), 27-day differentiated gonadal organoids+hCG (red line; mean±SEM; n=3) and hCG alone (grey line; mean±SEM; n=4), are presented.

In other examples, a spayed or neutered animal, such as a dog, cat, or other pet, may receive a slow-release form of chorionic gonadotrophin (CG) and may be administered to the animal at least 4 d prior to administering the therapeutically effective amount of the gonadal organoids to the animal. Various examples of slow-release CG may include an implantable device, a transdermal patch, depot suspension, or the like. This slow-release CG serves as the pre-treatment to prepare the environment to accept the gonadal organoids and allow immediate integration. Following the CG pre-treatment, the gonadal organoids can be administered, and the slow-release CG continues to facilitate integration and functionality of the gonadal organoids into the animal. Following a pre-determined delay, a blood sample may be analyzed for integration and/or functionality of the gonadal organoids and the slow-release CG may be removed if present.

### Conclusion

This example demonstrates the generation of functional gonadal organoids that can be injected into non-gonadal sites provides a base technology for the restoration of hormone balance in both intact and oophorectomized/orchiectomized individuals.

### Example 9

The above methods in Examples 1-8 can be utilized to rebalance the HPG axis and reverse or prevent dyotic signaling in tissues, thereby allowing for a more conducive environment for innate tissue regeneration or regeneration aided by treatment with donor cells. The methods from Examples 1-8 can be performed on patients, circulating and tissue sex hormone concentrations measured to confirm the HPG axis is rebalanced and that dyotic signaling has decreased, prior to the injection of donor cells into specific tissues or the circulation, and tissue regeneration and function monitored. As one example, the method of Example 1 can be used to decrease dyotic signaling to the brain, and donor cells (e.g. neural stem cells, iPS cells or differentiated neural cells) injected into a dysfunctioning region(s) of the brain. As another example, the methods in Example 8 can be utilized to rebalance the HPG axis and reverse or prevent dyotic signaling in tissues, and donor cells (e.g. ADSC, MSC, stem cells differentiated towards or into chondrocytes) then injected into osteoarthritic joints.

### Example 10

The patient is pre-treated with agents to lower dyotic signaling, such as GnRH agonists/antagonists and/or sex steroid supplementation (e.g. testosterone in males; estradiol and progesterone in females), prior to treatment with donor cells as outlined in Examples 1-8 to aid in the repopulation of gonadal cells.

Pre-treatment of patients described above is performed prior to the injection of donor cells into the gonads, non-gonadal tissues or the circulation, and tissue regeneration and function monitored.

### Example 11

These techniques can be used to treat hypogonadotropic hypogonadism (secondary hypogonadism), a condition characterized by hypogonadism due to an impaired secretion of gonadotropins, including FSH and LH, by the pituitary gland in the brain, and in turn decreased gonadotropin levels and a resultant lack of sex steroid production. Pituitary cell types such as gonadotrophs, corticotrophs, thyrotrophs, lactotrophs and adipose generated by way of Examples 1-3, 5-8, and from pituitary tissue, can be cultured to produce sufficient cell numbers to be injected into the pituitary, and/or injected into the circulation, and/or other tissues of the body to rebalance the HPG axis as described in Examples 1-8 with or without pre-treatment of patients described in Examples 9 and 10. Circulating and tissue sex hormone concentrations measured as described in Example 1 are performed to confirm rebalancing of the HPG axis. Conditions and diseases treated by this method include secondary congenital forms of hypogonadism (hypogonadotropic hypogonadism): Kallman syndrome, isolated GnRH deficiency, isolated LH deficiency, Prader-Willi syndrome, Turner syndrome, and Laurence-Moon-Biedl syndrome; and secondary acquired forms of hypogonadism: pituitary tumors and infarct, trauma, mumps, traumatic brain injury, children born to mothers who had ingested the endocrine disruptor diethylstilbestrol, opioid induced androgen deficiency (resulting from the prolonged use of opioid class drugs, e.g. morphine, oxycodone, methadone, fentanyl, hydromorphone), anabolic steroid-induced hypogonadism craniopharyngioma, hyperprolactemia (1° & 2°), hemochromatosis and neurosarcoid.

### Example 12

The above techniques also can be used to treat other dysregulated hormone axes of the body, including conditions and diseases that dysregulate the hypothalamic-pituitary-adrenal axis (e.g. adrenal insufficiency, Cushing's syndrome, Addison disease), alimentary system hormone axes, placental hormone axes, calcium regulatory axes, salt regulatory axes, thermoregulatory axes and thyroid hormone axes.

### Example 13

The above techniques in Examples 1-12 can be used to treat animals such as stud bulls or horses, pets and members of rare and endangered species in order to restore hormone balance and improve or maintain health and lifespan.

### Example 14: Proof of Concept Studies Assessing the Efficacy and Safety of Hormone-Producing Organoids for Reversing Hypogonadism and Increasing Healthy Longevity

Physiologic function requires continuously varying levels of many sex hormones—patterns best generated by intact, self-regulated glandular tissues. Therefore fully functional hormone-producing organoid (HPO) therapies that can be administered into non-gonadal tissues to restore circulating testicular hormones were developed. This biologic is a personalized therapeutic approach for restoring hormone balance in men with hypogonadism, including intact men over 50 and men who are castrated. A pre-clinical study was conducted to evaluate the sex hormone-producing organoid therapies for their capacity to 1) restore circulating sex hormone concentrations in a castrated rodent model of hypogonadism, and 2) assess impacts on longevity (Example 14 below). This biologic is a rat hormone-producing organoid (rHPO).

This example investigated the restoration of hormone balance in a castrated rat model of hypogonadism following treatment with rat hormone-producing organoids. As will be discussed in detail below, the testing demonstrated notable lifespan extension.

### Methods

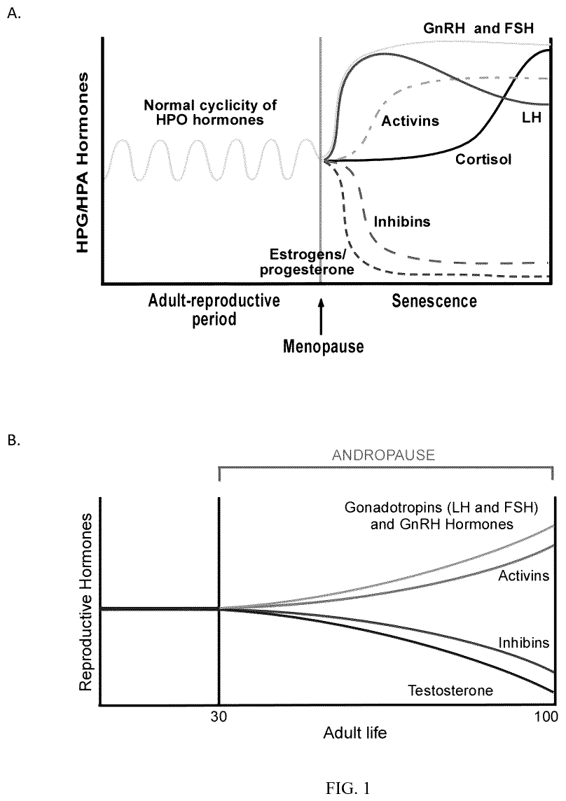

Generation of rat-hormone producing organoids (rHPO) for transplantation. Rat-hormone producing organoids were generated and injected into rats generally following the schematic shown in FIG. 1. Specifically, rat bone marrow-derived mesenchymal stem cells (rBM-MSCs) were isolated from the long-bone marrow of a neonatal male Fischer 344 rat, expanded in culture, differentiated into hormone-producing organoids (HPOs), and injected subcutaneously into the dorsal neck (back of neck) of adult male Fischer 344 rats after castration (see FIG. X, model of hypogonadism).

Cell bank. rBM-MSCs were isolated from the long-bone marrow of a neonatal male Fischer 344 rat. Using a 1 mL syringe with a 27-gauge needle, DMEM/F-12 without phenol red (JangoCell, Fitchburg, WI, USA) was flushed through the bone to expel marrow, which was then gently triturated with a pipette. Red blood cell lysis buffer (1 mL; eBioscience, Thermo Fisher Scientific, Waltham, MA, USA) was added to the marrow suspension for 5 min. at 4° C.; lysis was stopped by adding Quench medium (DMEM/F-12 containing 10% FBS). The mixture was centrifuged for 5 minutes at 350 g, the supernatant removed, and the pellet resuspended in DMEM/F-12 without phenol red, recentrifuged, and the supernatant removed. The pellet was then resuspended and plated in mesenchymal stem cell medium (MSCM; ScienCell Laboratories, Carlsbad, CA, USA) supplemented with 5% FBS, MSC Growth Supplement (MSCGS), and penicillin/streptomycin (ScienCell Laboratories) in T-flasks (TPP Techno Plastic Products AG, Trasadingen, Switzerland) and cultured at 37° C. with 5% CO₂. Upon reaching 80-90% confluence, rBM-MSCs were passaged every 5-7 days. When the required cell number was reached, cells were washed in PBS, detached with 0.25% trypsin-EDTA (Gibco, Thermo Fisher Scientific) for 5 minutes, and neutralized with culture medium. The cell suspension was centrifuged at 350 g for 5 minutes, the supernatant discarded, and the pellet resuspended in NutriFreez D10 (Sartorius, Göttingen, Germany) and banked in liquid nitrogen.

Differentiation medium. Guided by transcription factors required for macrophage, Leydig, peritubular myoid, Sertoli, and spermatogonial stem cell (SSC) lineages (SF1, WT1, GATA-1, GATA-4, DMRT1, and c-MYC), a literature review was combined with preliminary in vitro screening of >200 hormone and growth-factor permutations—each acting through these factors—to formulate a medium that drives stem cells into sex-hormone-producing organoids. The resulting formulation, Cellular Differentiation Medium-3 (CDM-3), contains defined developmental hormones and growth factors and enables neonatal rBM-MSCs to differentiate into organoids comprising six key cell types that support robust hormone production. CDM-3 also supports incorporation of vascular endothelial cells and vascular endothelial growth factor (VEGF) within organoids, promoting in vivo angiogenesis and anastomosis necessary for sustained perfusion.

Hormone-Producing Organoid Production. Early-passage rBM-MSCs were thawed into T-flasks and expanded in a MSC Medium. Cells were then seeded onto Plastic Plus microcarriers (Corning Incorporated, Corning, NY, USA) and cultured in 1 L glass spinner flasks (Corning Incorporated) for large-scale expansion. At 80-90% confluence, cells were dissociated as above, pooled to 2×10 million cells/mL in the MSC Medium supplemented with the ROCK inhibitor H1152 (AdipoGen Life Sciences, San Diego, CA, USA), and transferred to a spinner flask agitated at 70 rpm in a humidified incubator at 37° C. with 5% CO₂. Once spheroids were visible (1-2 days), the medium was replaced with CDM-3 to generate rHPOs. Cultures were maintained in CDM-3 with 50% medium exchange every 3 days for a total of 11-28 days. On the day of rat injection, the rHPOs were allowed to settle in the spinner flasks, collected, washed in PBS, resuspended in DMEM/F-12 (Gibco, Thermo Fisher Scientific), and loaded into 1 mL syringes with 22-gauge needles (BD, Franklin Lakes, NJ, USA).

Flow cytometry characterization of organoids. Organoids generated as described were dissociated into single-cell suspensions and analyzed by flow cytometry. Data were acquired on an Attune NxT Flow Cytometer (Thermo Fisher Scientific, Waltham, MA, USA) with ≥50,000 events collected per marker. Viable cells were identified using a fixable Live/Dead viability dye (Thermo Fisher Scientific). Gates were set on singlets and live cells. Positive populations were defined using fluorescence-minus-one (FMO) controls for each fluorochrome. Antibodies (listed from least to most differentiated stage for each lineage):
Testicular macrophage: MHC class II (mouse IgG1; Thermo Fisher Scientific, Waltham, MA, USA); CD64 (mouse IgG1, PerCP-eFluor 780; Thermo Fisher Scientific).
Leydig: PDGF-Rα (mouse IgG1, biotin-conjugated; Thermo Fisher Scientific); CYP11A1 (rabbit polyclonal; Abcam, Cambridge, UK); 17β-HSD (mouse IgG1; Santa Cruz Biotechnology, Dallas, TX, USA); LHR/LHCGR (mouse IgG1; Novus Biologicals, Centennial, CO, USA).
Myoid: α-SMA (mouse IgG2a; Thermo Fisher Scientific); CD11b (mouse IgG1; Thermo Fisher Scientific); MYH11 (rabbit monoclonal; Abcam).
Sertoli: SOX9 (rabbit polyclonal; LifeSpan BioSciences, Seattle, WA, USA); androgen receptor (mouse IgG1, biotin-conjugated; Thermo Fisher Scientific); inhibin-βB (mouse IgG2; Santa Cruz Biotechnology); FSHR (mouse IgG1; Novus Biologicals); AMH (rabbit monoclonal; Abcam).
Spermatogonial stem cell (SSC): PLZF (rabbit monoclonal; Cell Signaling Technology, Danvers, MA, USA); GFRα1 (mouse IgG1; Santa Cruz Biotechnology); SSEA-4 (mouse monoclonal; MilliporeSigma, Burlington, MA, USA).
Vascular: VEGF (rabbit polyclonal; Thermo Fisher Scientific); VEGFR2/KDR (rabbit monoclonal; Cell Signaling Technology).

Rat injection, blood collection, and hormone analyses. Adult male Fischer rats (4-6 months, n=8) were castrated, or castrated and injected subcutaneously >1-month post-castration into the dorsal neck (under aseptic conditions) with either saline or a single dose of rHPOs. Prior to injection rats were treated with hCG, and then treated up to day 15 post-rHPO injection, according to the schedule described in Example 8. Cell suspensions in DMEM/F-12 (Gibco, Thermo Fisher Scientific) were delivered with syringes fitted with 22 G, 1-inch needles (BD, Franklin Lakes, NJ, USA). Across experiments, doses ranged from 10-40 million cells/mL per injection. Blood (2 mL) was collected from the saphenous vein of rats under anesthesia, and the plasma removed at the following timepoints: pre-castration, post-castration, pre-injection (Day 0), Day 4, Day 7, and then every 1-2 weeks for 8 weeks and every 4-6 weeks thereafter. Plasma sex steroids (progesterone, dehydroepiandrosterone [DHEA], testosterone, estradiol) were quantified by electrochemiluminescence using a 4-Spot Custom Steroid Hormone Panel on the MSD platform (Meso Scale Diagnostics, Rockville, MD, USA).

Longevity analysis. Animals were observed at least daily and followed until death; lifespan was the age at natural death or IACUC-defined humane endpoint, with survivors right-censored. Kaplan-Meier curves were generated in GraphPad Prism; per-cohort mean, SD, SEM, minimum, and maximum lifespans were tabulated. An intact Fischer 344 rat survival curve from literature (Smith et al. J Gerontol A Biol Sci Med Sci. 2010 May; 65A(5):468-474) was included in survival analyses for context only and was not part of the testing in this example.

### Results

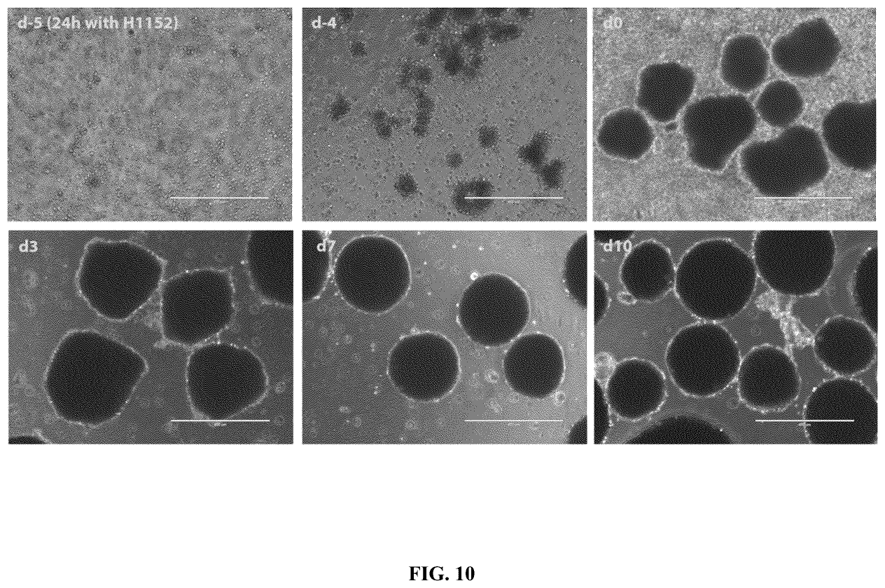

After expansion, rBM-MSCs were dissociated, pooled and then transferred to a 1 L spinner flask at 70 rpm at 2 million cells/mL in MSC Medium containing ROCK inhibitor H1152 (day −5). When early spheroids became visible (1-2 days later), the medium was replaced with CDM-3 to initiate differentiation (day 0). Cultures were maintained in CDM-3 with 50% medium exchange every 3 days for 11-28 days. Morphology (FIG. 10) shows single cells and loose aggregates at d−5 and d−4, compact spheroids at d0, and progressive growth and rounding through d3, d7, and d10 of differentiation.

### Flow Cytometry

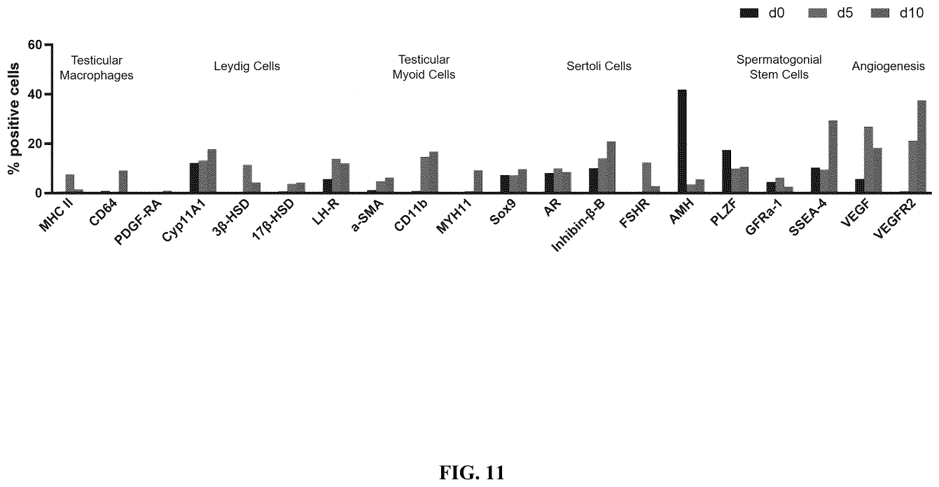

CDM-3 produced multi-lineage testicular organoids with time-structured biomarker shifts (FIG. 11). Testicular macrophage markers MHC II and CD64 remained low. Leydig markers, with the exception of PDGF-RA, increased during differentiation. Testicular myoid markers showed α-SMA increasing slightly over time, CD11b increasing substantially and remaining elevated, and MYH11 rising at d9. Sertoli markers showed SOX9 and AR remaining elevated, inhibin-β-B increasing over time, FSHR rising at d5 then decreasing at d10, and AMH decreasing sharply at d5 and remaining low at d10. SSC markers showed PLZF and GFRα-1 remaining stable, while SSEA-4 increased modestly at d10. Vascular markers showed both VEGF and VEGFR2increasing sharply.

### Plasma Hormone Analysis

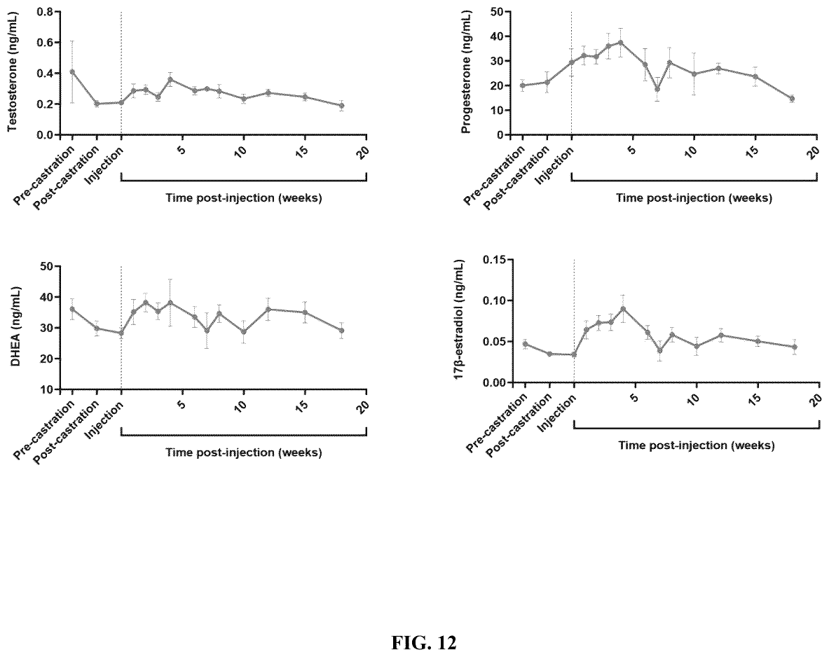

Across animals, the plasma hormone profiles (FIG. 12) indicate sustained secretion of multiple steroids by the implanted organoids for 3-4 months after injection. Castration reduced circulating testosterone to near-baseline nadirs, followed by a modest post-implantation rise that peaked within 3-5 weeks and then trended downward but remained detectable through 18 weeks. Progesterone increased after implantation, reaching a broad peak around weeks 3-6, and stabilized at intermediate levels thereafter. DHEA remained within a 20-40 ng/mL band with mild fluctuations and no clear directional trend. 17β-estradiol showed a small rise by 3-5 weeks post-injection and then plateaued at low concentrations.

### Lifespan Analysis

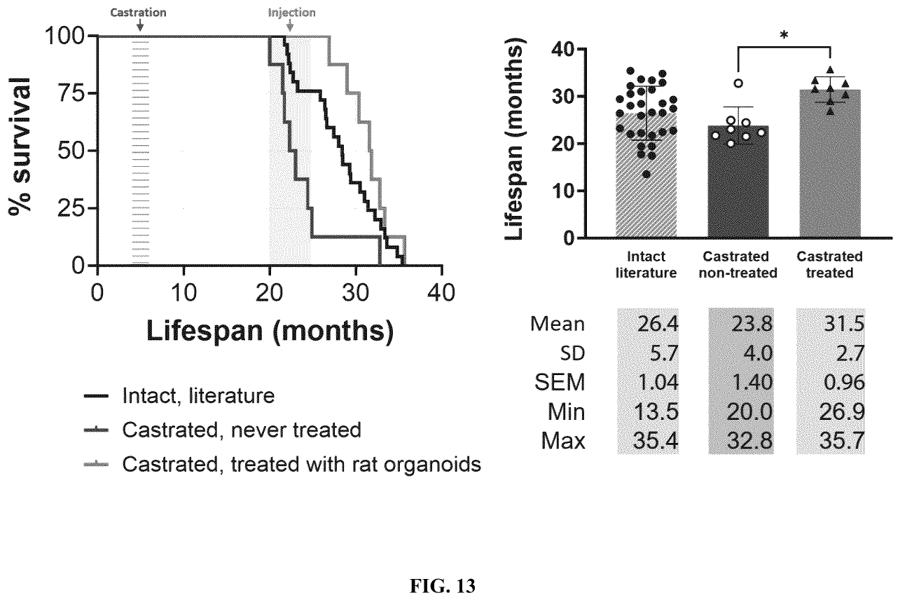

Castration shortened the lifespan of male Fischer 344 rats relative to intact Fischer 344 rats reported in the literature (Smith et al. 2010). Conversely, rHPO treatment shifted the survival curve rightward and increased mean lifespan versus the castrated untreated group (FIG. 13). The mean gain in longevity was equivalent to 7.7 months compared to the castrated untreated rats, and 5.1 months compared to the intact rat literature. Maximum longevity was modestly decreased in castrated untreated rats compared to intact and castrated treated rats.

### Discussion

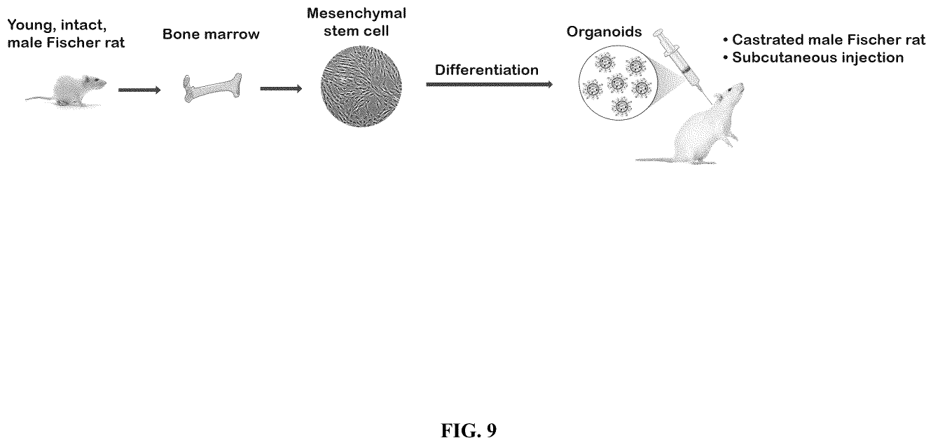

The testing in this example confirms a scalable protocol to aggregate rBM-MSCs into spheroids, subsequently compact these into dense spheroids, and then differentiate them into organized functional organoids (FIG. 9) that persistently secrete multiple hormones in vivo following subcutaneous implantation. Flow cytometry demonstrated the presence of the major testicular cellular lineages, including Sertoli, Leydig, peritubular myoid, vascular/endothelial, macrophage, and SSC-associated markers, following directed differentiation in CDM-3 (FIG. 10). In male castrated Fischer 344 rats, plasma testosterone, progesterone, estradiol, and DHEA concentrations rose from post-castration nadirs to sustained levels over 3-4 months (FIG. 11) and correlated with a 33% increase in longevity relative to castrated-untreated animals (FIG. 12).

### Validation of Hormone-Producing Organoid Production and Applications for Reversing Hypogonadism

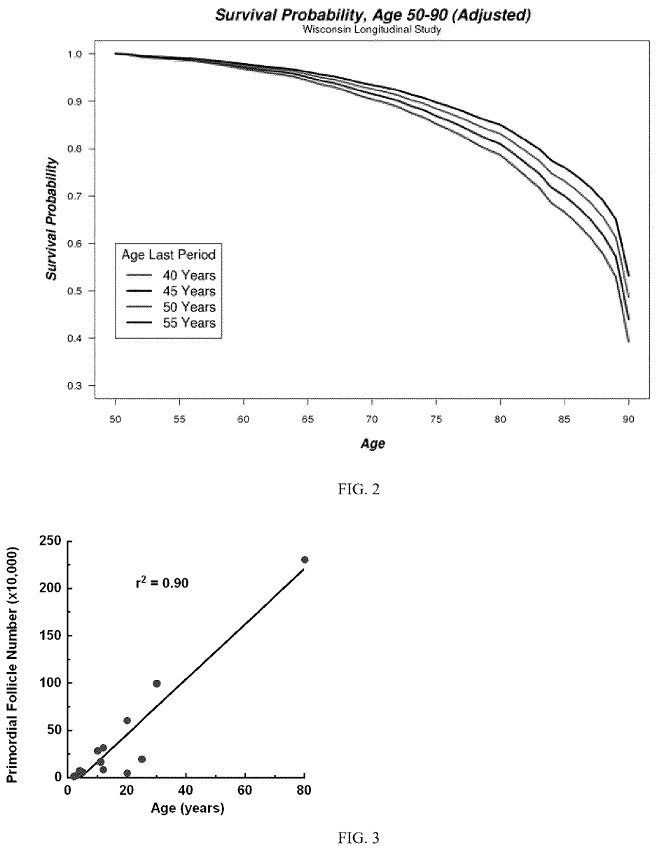

HPO's generated in the testing shown in this Example contain critical cell types responsible for normal gonadal endocrine function. The testicular cellular biomarker set suggests CDM-3 drives multilineage organization supportive of steroidogenesis and protein hormone expression: PDGF-Rα and CYP11A1 indicate Leydig commitment; SOX9, FSHR, AR, and inhibin-βB indicate Sertoli maturation; VEGF/VEGFR2 imply angiogenic capacity needed for perfusion; PLZF/GFRA1/SSEA-4 are consistent with an undifferentiated germline niche (FIG. 2). The in vivo steroid profiles are consistent with functional steroidogenic circuitry rather than isolated enzyme expression. Compared with prior approaches, which often fail due to poor vascular access or incomplete paracrine support, the observed endothelial signals and macrophage/myoid presence in these organoids (FIG. 10) plausibly address those deficits by improving vascular ingrowth and extracellular matrix dynamics. The defined CDM-3 formulation and spinner-based scale-up provide a practical path toward biomanufacturing on scale.

To test the HPO's in vivo, a castration model, which approximates endocrine dyscrasia from gradual, natural transitions (menopause; age-related androgen decline), abrupt surgical loss (oophorectomy; orchiectomy), and therapeutic suppression (GnRH analogs/antagonists, chemotherapy, pelvic irradiation, androgen-deprivation therapy, aromatase inhibitors), each characterized by altered circulating reproductive hormones, was utilized. Months-long secretion of gonadal hormones (FIG. 11) without overt procedure-related mortality differences in the early phase was observed. This is consistent with tolerability of subcutaneous dorsal-neck implantation.

### Applications of Hormone-Producing Organoids for Increasing Healthy Longevity

Sustained multi-hormone secretion after a single implantation supports the concept of extra-gonadal endocrine support in hypogonadal states, including surgical and age-related contexts. Indeed, in castrated Fischer 344 rats, a single subcutaneous dose of rHPO significantly extended health span by 32% compared to untreated castrated controls, and 19% compared to intact controls (FIG. 12). These data suggest reversing endocrine dyscrasia can shift survival distributions.

The survival right-shift in treated rats suggests systemic benefits related to restoration of hormone balance required for optimal somatic tissue function.

For translational context only, using 13.8 rat days≈1 human year (Sengupta 2013), the observed mean survival gain of 7.7 months between castrated treated versus castrated untreated rats equates to an additional≈16.9 healthy human years of life. Similarly, the mean survival gain of 5.1 months between intact versus castrated treated rats equates to an additional≈11.3 healthy human years of life. No short-term local injection related or long-term, safety issues were reported for the rats.

### Conclusion

CDM-3-guided, spinner flask-generated organoids reproduce essential testicular cell interactions, sustain multi-hormone secretion in vivo, and improve survival in castrated rats relative to untreated controls. The restoration of circulating gonadal sex hormones back to youthful concentrations significantly increases the lifespan of castrated male Fischer 344 rats (32%) as compared to untreated castrated controls. These findings support extra-gonadal organoid therapy as a plausible route to hormonal reconstitution in those with endocrine dyscrasia as a result of induced hypogonadism or following menopause or andropause. Furthermore, since death is the result of a disease of aging (tissue dysfunction), these data indicate that restoring hypothalamic-pituitary-gonadal (HPG) hormone balance mitigates diseases of aging, since treated animals lived significantly longer. These results provide support for the Reproductive Cell-Cycle Theory of Aging (Bowen and Atwood, 2004) that posits post-reproductive endocrine dyscrasia drives cellular and tissue degeneration and dysfunction, leading to tissue failure, disease, and ultimately death.

The HPOs of the instant disclosure provide multiple advantages over current hormone replacement therapy options, including: 1) a personalized medicine approach to recreating HPG axis hormonal balance according to each individual's requirements; 2) a more complete rebalancing of sex hormones, including both sex steroids and protein sex hormones; 3) enabling appropriate hormone specific production on a minute-to-minute, hour-to-hour and day-to-day basis; 4) a long-term (semi-permanent) solution to restoring circulating hormone levels; and 5) substantial health and lifespan advantages over current pharmaceutical replacement therapies.

While various embodiments of the present invention have been described above, it should be understood that they have been presented by way of example only, and not by limitation. For example, the present invention is not limited to the stem or differentiated cells illustrated or described, the methods of injection, the hormones produced by the cells, or the injected tissues illustrated or described. In another example, although some cells and techniques described herein are related to humans, the present invention is not limited to humans, but rather, includes all reproductively viable organisms. As such, the breadth and scope of the present invention should not be limited to any of the above-described exemplary embodiments, but should be defined only in accordance with the following claims and their equivalents.

Aspects: Examples of numbered aspects and further aspects of the invention are shown below:
Aspect 1 is a method of treating male hypogonadism in a subject in need thereof comprising administering to the subject testicular organoids generated from isolated male progenitor cells. In certain preferred embodiments, aspect 1 is a method of treating male hypogonadism in a subject in need thereof that has not been pre-treated with CG comprising administering to the subject testicular organoids generated from isolated male progenitor cells

Aspect 2 is the method of aspect 1, wherein the subject is not being administered testosterone replacement therapy.

Aspect 3 is the method of aspects 1 or 2, wherein the hypogonadism is a result of andropause or testosterone deficiency syndrome (TDS), or congenital or acquired primary hypogonadism.

Aspect 4 is the method of any one of aspects 1 to 3, wherein the subject is a male mammal.

Aspect 5 is the method of aspect 4 wherein the subject is a human.

Aspect 6 is the method of aspect 5, wherein the human is 50 years old or older.

Aspect 7 is the method of any one of aspects 1 to 6, wherein method comprises injection of the organoids.

Aspect 8 is the method of any one of aspects 1 to 7, wherein the male progenitor cells are isolated from the subject.

Aspect 9 is the method of any one of aspects 1 to 8, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

Aspect 10 is the method of any one of aspects 1 to 9, wherein the method restores hormone balance.

Aspect 11 is the method of cany one aspect 1 to 10, wherein the method does not further comprise pre-treating the subject with a chorionic gonadotrophin (CG).

Aspect 12 is the method of any one of aspects 1 to 11, wherein the method further comprises generating the testicular organoids by expanding isolated male progenitor cells, generating a spheroid under dynamic culture conditions, and culturing the spheroid under dynamic culture conditions to generate the testicular organoid.

Aspect 13 is the method of aspect 12, wherein the method comprises culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

Aspect 14 is the method of any one of aspects 1 to 13, wherein the progenitor cells are adult-derived or umbilical cord-derived mesenchymal stem cells, or adipose-derived stem cells (ADSC).

Aspect 15 is a method of generating a testicular organoid, the method comprising: (a) generating a spheroid from isolated male progenitor cells under dynamic conditions; and (b)
culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

Aspect 16 is the method of aspect 15, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

Aspect 17 is the method of aspect 16, wherein the testicular organoid comprises at least 5-30% cells expressing PDGF-Rα, Cyp11A1, 3β-HSD, 17β-HSD, and LHCGR, about 5-50% of cells expressing α-SMA and CD11b, about 5-50% of cells expressing MHC CII and CD64, at least 5-30% of cells expressing Sox9, AR, inhibin β-B, and FSHR, and about 5-30% of cells expressing VEGF and VEGFR2, and cells expressing about 3-15% GFRA1.

Aspect 18 is the method of any one of aspects 15 to 17, wherein the progenitor cells are adult-derived or umbilical cord-derived mesenchymal stem cells, or adipose-derived stem cells (ADSC).

Aspect 19 is the method of aspect 18, wherein the method comprises culturing the cells for at least 7 days or for about 14 days to about 27 days.

Aspect 20 is the method of aspect 19, wherein the method further comprises culturing the progenitor cells in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA.

Aspect 21 is a method of increasing health span or longevity in a male subject in need thereof comprising administering to the subject testicular organoids generated from isolated male progenitor cells.

Aspect 22 is the method of aspect 21, wherein the male subject is a male mammal or human.

Aspect 23 is the method of aspect 22, wherein the human is 50 years old or older.

Aspect 24 is the method of any one of aspects 21-23, wherein method comprises injection of the organoids.

Aspect 25 is the method of any one of aspects 21-24, wherein the male progenitor cells are isolated from the subject.

Aspect 26 is the method of any one of aspects 21-25, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

Aspect 27 is the method of any one of aspects 21-28, wherein the method does not further comprise pre-treating the subject with a chorionic gonadotrophin (CG).

Aspect 28 is the method of any one of aspects 21-27, wherein the method further comprises generating the testicular organoids by expanding isolated male progenitor cells, generating a spheroid under dynamic culture conditions, and culturing the spheroid under dynamic culture conditions to generate the testicular organoid.

Aspect 29 is the method of aspect 28, wherein the method comprises culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

Aspect 30 is the method of aspect 21, wherein the progenitor cells are adult-derived or umbilical cord-derived mesenchymal stem cells, or adipose-derived stem cells (ADSC).

Aspect 31 is a method of rebalancing the HPG axis in a male subject in need thereof comprising administering to the subject testicular organoids generated from isolated male progenitor cells.

Aspect 32 is the method of aspect 31, wherein the male subject is a male mammal or human.

Aspect 33 is the method of aspect 32, wherein the human is 50 years old or older.

Aspect 34 is the method of aspect 31, wherein method comprises injection of the organoids.

Aspect 35 is the method of any one of aspects 31-34, wherein the male progenitor cells are isolated from the subject.

Aspect 36 is the method of any one of aspects 31-35, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

Aspect 37 is the method of any one of aspects 31-36, wherein the method does not further comprise pre-treating the subject with a chorionic gonadotrophin (CG).

Aspect 38 is the method of any one of aspects 31-37, wherein the method further comprises generating the testicular organoids by expanding isolated male progenitor cells, generating a spheroid under dynamic culture conditions, and culturing the spheroid under dynamic culture conditions to generate the testicular organoid.

Aspect 39 is the method of aspect 38, wherein the method comprises culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

Aspect 40 is the method of any one of aspects 31-39, wherein the progenitor cells are adult-derived or umbilical cord-derived mesenchymal stem cells, or adipose-derived stem cells (ADSC).

Aspect 41 is the method of any one of aspects 31-40, wherein the subject is suffering from an age-related disease.

Aspect 42 is the method of slowing or halting diseases of aging in a male subject in need thereof comprising administering to the subject testicular organoids generated from isolated male progenitor cells.

Aspect 43 is the method of aspect 42, wherein the male subject is a male mammal or human.

Aspect 44 is the method of aspects 42 and 43, wherein method comprises injection of the organoids.

Aspect 45 is the method of any one of aspects 42-44, wherein the method does not further comprise pre-treating the subject with a chorionic gonadotrophin (CG).

Aspect 46 is the method of any one of aspects 42-45, wherein the human is 50 years old or older.

Aspect 47 is the method of any one of aspects 42-46, wherein method comprises injection of the organoids.

Aspect 48 is the method of any one of aspects 42-47, wherein the male progenitor cells are isolated from the subject.

Aspect 49 is the method of any one of aspects 42-48, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

Aspect 50 is the method of any one of aspects 42-49, wherein the method further comprises generating the testicular organoids by expanding isolated male progenitor cells, generating a spheroid under dynamic culture conditions, and culturing the spheroid under dynamic culture conditions to generate the testicular organoid.

Aspect 51 is the method of aspect 50, wherein the method comprises culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

Aspect 52 is the method of any one of aspects 42-50, wherein the progenitor cells are adult-derived or umbilical cord-derived mesenchymal stem cells, or adipose-derived stem cells (ADSC).

Further Aspect 1. A method of improving integration of gonadal organoids introduced to a subject, the method comprising: pre-treating the subject with a chorionic gonadotrophin (CG); administering a therapeutically effective amount of the gonadal organoids to the subject; and administering the CG to the subject to maintain LHCG receptor expression and signaling, the CG being administered periodically over time to cause a fluctuation of a concentration of the CG in the subject, or the CG being administered at a constant low CG concentration.

Further Aspect 2. The method according to further aspect 1, wherein the pre-treatment includes administering the CG at least 4 days prior to the administration of the gonadal organoids.

Further Aspect 3. The method according to further aspect 2, wherein the CG is administered periodically at least 4 days prior to gonadal organoid injection to maintain LHCG receptor expression and signaling.

Further Aspect 4. The method according to further aspect 2, wherein the pre-treatment of CG is administered 7, 5, 3, and 1 day before, or 7, 4 and 1 days before, the administration of the gonadal organoids.

Further Aspect 5. The method according to any one of further aspects 1-4, wherein the CG is administered periodically to maintain LHCG receptor expression and signaling for 2-4 weeks following the administration of the gonadal organoids.

Further Aspect 6. The method according to further aspect 5, wherein the CG is administered every other day for 2-4 weeks, or every 3 days for 2-4 weeks, or every 4 days for 2-4 weeks, following the administration of the gonadal organoids.

Further Aspect 7. The method according to any one of further aspects 1-6, wherein the CG is administered on the day of the administration of the gonadal organoids and then periodically for 2-4 weeks.

Further Aspect 8. The method according to any one of further aspects 1-7, further comprising: plating adult-derived or umbilical cord-derived mesenchymal stem cells (MSCs) in CDM-3.4 differentiation media to generate the gonadal organoids.

Further Aspect 9. The method according to further aspect 8, further comprising: growing cells isolated from bone marrow or umbilical cord in mesenchymal stem cell media to generate the MSCs.

Further Aspect 10. The method according to further aspect 8, further comprising: challenging the gonadal organoids individually or with a combination of LH, FSH, and/or hCG to determine a hormone characterization of the gonadal organoids.

Further Aspect 11. The method according to any one of further aspects 1-10, wherein the CG is a human chorionic gonadotrophin (hCG).

Further Aspect 12. The method according to any one of further aspects 1-10, wherein the CG is an animal-specific chorionic gonadotropin.

Further Aspect 13. The method according to any one of further aspects 1-12, wherein 10-10,000 international units (IU) per kilogram (kg) of the CG is administered.

Further Aspect 14. The method according to any one of further aspects 1-12, wherein 50-200 IU/kg of the CG is administered.

Further Aspect 15. The method according to further aspect 11, wherein about 100 IU/kg of the CG is administered.

Further Aspect 16. The method according to any one of further aspects 1-15, further comprising: plating adipose-derived stem cells (ADSCs) in CDM-3.4 differentiation media to generate the gonadal organoids.

Further Aspect 17. The method according to further aspect 16, further comprising: growing cells isolated from adipose tissue in adipose-derived stem cell media to generate the ADSCs.

Further Aspect 18. The method according to further aspect 16, further comprising: challenging the gonadal organoids with one or more of LH, FSH, and hCG to determine a hormone characterization of the gonadal organoids.

Further Aspect 19. The method according to any one of further aspects 1-18, further comprising: implanting a slow-release device in the subject, the slow-release device being configured to administer the CG to the subject for 2-5 weeks, wherein the slow-release device is implanted one week prior to administering the therapeutically effective amount of the gonadal organoids to the subject to pre-administer the CG and the slow-release continues to administer the CG following administration of the gonadal organoids.

Further Aspect 20. A method of improving functionality of gonadal organoids introduced to a subject, the method comprising: pre-treating the subject with a chorionic gonadotrophin (CG); administering a therapeutically effective amount of the gonadal organoids to the subject; and administering the CG to the subject to maintain LHCG receptor expression and signaling, the CG being administered periodically over time to cause a fluctuation of a concentration of the CG in the subject, or the CG being administered at a constant low CG concentration.

Further Aspect 21. The method according to claim 20, wherein the pre-treatment includes administering the CG at least 4 days prior to the administration of the gonadal organoids.

Further Aspect 22. The method according to further aspect 21, wherein the CG is administered periodically at least 4 days prior to gonadal organoid injection to maintain LHCG receptor expression and signaling.

Further Aspect 23. The method according to further aspect 21, wherein the pre-treatment of CG is administered 7, 5, 3, and 1 day before, or 7, 4 and 1 days before, the administration of the gonadal organoids.

Further Aspect 24. The method according to further aspect 20, wherein the CG is administered periodically to maintain LHCG receptor expression and signaling for 2-4 weeks following the administration of the gonadal organoids.

Further Aspect 25. The method according to further aspect 24, wherein the CG is administered every other day for 2-4 weeks, or every 3 days for 2-4 weeks, or every 4 days for 2-4 weeks, following the administration of the gonadal organoids.

Further Aspect 26. The method according to further aspect 20, wherein the CG is administered on the day of the administration of the gonadal organoids and then periodically for 2-4 weeks.

Further Aspect 27. The method according to any one of further aspects 20-26, further comprising plating adult-derived or umbilical cord-derived mesenchymal stem cells (MSCs) in CDM-3.4 differentiation media to generate the gonadal organoids.

Further Aspect 28. The method according to further aspect 27, further comprising growing cells isolated from bone marrow or umbilical cord in mesenchymal stem cell media to generate the MSCs.

Further Aspect 29. The method according to further aspect 27, further comprising challenging the gonadal organoids individually or with a combination of LH, FSH, and hCG to determine a hormone characterization of the gonadal organoids.

Further Aspect 30. The method according to any one of further aspects 20-29, wherein the CG is a human chorionic gonadotrophin (hCG).

Further Aspect 31. The method according to any one of further aspects 20-29, wherein the CG is an animal-specific chorionic gonadotropin.

Further Aspect 32. The method according to any one of further aspects 20-31, wherein 10-10,000 international units (IU) per kilogram (kg) of the CG is administered.

Further Aspect 33. The method according to further aspect 32, wherein 50-500 IU/kg of the CG is administered.

Further Aspect 34. The method according to further aspect 33, wherein about 100 IU/kg of the CG is administered.

Further Aspect 35. The method according to any one of further aspects 20-34, further comprising plating adipose-derived stem cells (ADSCs) in CDM-3.4 differentiation media to generate the gonadal organoids.

Further Aspect 36. The method according to further aspect 35, further comprising: growing cells isolated from adipose tissue in adipose-derived stem cell media to generate the ADSCs.

Further Aspect 37. The method according to further aspect 35, further comprising challenging the gonadal organoids with one or more of LH, FSH, and hCG to determine a hormone characterization of the gonadal organoids.

Further Aspect 38. The method according to any one of further aspects 20-37, further comprising: implanting a slow-release device such as a polymer matrix in the subject, the slow-release device being configured to administer the CG to the subject for 2-5 weeks, wherein the slow-release device is implanted one week prior to administering the therapeutically effective amount of the gonadal organoids to the subject to pre-administer the CG and the slow-release continues to administer the CG following administration of the gonadal organoids.

Further Aspect 39. A method of generating a testicular organoid, the method comprising: generating a spheroid from isolated male progenitor cells; and culturing the spheroid in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

Further Aspect 40. The method of further aspect 39, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

Further Aspect 41. The method of further aspect 40, wherein the testicular organoid comprises at least 5-30% cells expressing PDGF-Rα, Cyp11A1, 3β-HSD, 17β-HSD, and LHCGR, about 5-50% of cells expressing α-SMA and CD11b, about 5-50% of cells expressing MHC CII and CD64, at least 5-30% of cells expressing Sox9, AR, inhibin β-B, and FSHR, and about 5-30% of cells expressing VEGF and VEGFR2, and cells expressing about 3-15% GFRA1.

Further Aspect 42. The method of any one of further aspects 39-41, wherein the progenitor cells are adult-derived or umbilical cord-derived mesenchymal stem cells, or adipose-derived stem cells (ADSC).

Further Aspect 43. The method of any one of further aspects 39-42, wherein the method comprises culturing the cells for at least 7 days or for about 14 days to about 27 days.

Further Aspect 44. The method of further aspect 39, wherein the method comprises culturing the progenitor cells in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA.

Further Aspect 45. A method of generating a testicular organoid, the method comprising culturing progenitor cells in a culture medium supplemented with a luteinizing hormone, follicle stimulating hormone, a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

Further Aspect 46. The method of further aspect 45, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

Further Aspect 47. The method of further aspect 46, wherein the testicular organoid comprises at least 5-30% cells expressing PDGF-Rα, Cyp11A1, 3β-HSD, 17β-HSD, and LHCGR, about 5-50% of cells expressing α-SMA and CD11b, about 5-50% of cells expressing MHC CII and CD64, at least 5-30% of cells expressing Sox9, AR, inhibin β-B, and FSHR, and about 5-30% of cells expressing VEGF and VEGFR2, and cells expressing about 3-15% GFRA1.

Further Aspect 48. The method of any one of further aspects 45-47, wherein the culturing comprises culturing the cells for at least 7 days or for about or 14 days to about 27 days.

Further Aspect 49. The method of any one of further aspects 45-48, wherein the progenitor cells are adult-derived or umbilical cord-derived mesenchymal stem cells or adipose-derived stem cells.

Further Aspect 50. The method of further aspect 39, wherein the method comprises culturing a spheroid comprising the progenitor cells.

Further Aspect 51. A method of generating an ovarian organoid, the method comprising: generating a spheroid from isolated female progenitor cells; and culturing the spheroid in a culture medium supplemented with a luteinizing hormone, follicle stimulating hormone, a thyroid hormone, human insulin-like growth factor 1 (IGF-1), human glial cell line-derived neurotrophic factor (GDNF), retinoic acid, testosterone, smoothened agonist (SAG), 8-bromoadenosine 3′,5′-cyclic adenosine monophosphate (8-Br-cAMP), and platelet-derived growth factor AA or platelet-derived growth factor AB to generate the ovarian organoid.

### References

Abd-Allah S H, Shalaby S M, Pasha H F, El-Shal A S, Raafat N, Shabrawy S M, Awad H A, Amer M G, Gharib M A, El Gendy E A, et al. 2013 Mechanistic action of mesenchymal stem cell injection in the treatment of chemically induced ovarian failure in rabbits. Cytotherapy 15 64-75.
Arakane M, Castillo C, Rosero M F, Penafiel R, Perez-Lopez F R & Chedraui P 2011 Factors relating to insomnia during the menopausal transition as evaluated by the Insomnia Severity Index. Maturitas 69 157-161.
Arantes-Oliveira N, Apfeld J, Dillin A & Kenyon C 2002 Regulation of life-span by germ-line stem cells in Caenorhabditis elegans. Science 295 502-505.
Atwood C & Vadakkadath Meethal S 2011a Gonadotropins and Progestogens: Obligatory Developmental Functions during Early Embryogenesis and their Role in Adult Neurogenesis, Neuroregeneration, and Neurodegeneration. In Hormones in Neurodegeneration, Neuroprotection and Neurogenesis, pp 305-319. Ed AGaS Mellon. Weinheim, Germany: WILEY-VCH Verlag GmbH & Co.
Atwood C & Vadakkadath Meethal S 2011b Human Embryonic Stem Cells as a Model System for Understanding Early Human Embryogenesis and Age-related Diseases. In Embryonic Stem Cells: The Hormonal Regulation of Pluripotency and Embryogenesis, pp 251-270. Ed CS Atwood. Rijeka, Croatia: InTech.
Atwood C S & Bowen R L 2007 Metabolic clues regarding the enhanced performance of elite endurance athletes from orchiectomy-induced hormonal changes. Medical hypotheses 68 735-749.
Atwood C S & Bowen R L 2011 The reproductive-cell cycle theory of aging: an update. Experimental Gerontology 46 100-107.
Atwood C S and Bowen R L 2015 The endocrine dyscrasia that accompanies menopause and andropause induces aberrant cell cycle signaling that triggers re-entry of post-mitotic neurons into the cell cycle, neurodysfunction, neurodegeneration and cognitive disease. Hormones and behavior 76 63-80.
Atwood C S, Meethal S V, Liu T, Wilson A C, Gallego M, Smith M A & Bowen R L 2005 Dysregulation of the hypothalamic-pituitary-gonadal axis with menopause and andropause promotes neurodegenerative senescence. J Neuropathol Exp Neurol 64 93-103.
AUA Guideline (2018). Evaluation and Management of Testosterone Deficiency. American Urological Association. https://doi.org/10.1016/j.juro.2018.03.115 (guideline page) Baert Y., et al. (2019). Testicular organoids: a new model to study the testis microenvironment and spermatogenesis. Andrology, 7 574-583. (overview/review).
Bagatell C J, Dahl K D & Bremner W J 1994 The direct pituitary effect of testosterone to inhibit gonadotropin secretion in men is partially mediated by aromatization to estradiol. Journal of andrology 15 15-21.
Belanger A, Candas B, Dupont A, Cusan L, Diamond P, Gomez J L & Labrie F 1994 Changes in serum concentrations of conjugated and unconjugated steroids in 40- to 80-year-old men. J Clin Endocrinol Metab 79 1086-1090.
Bhasin S. (2022). Age-Related Changes in the Male Reproductive System. In Endotext (NCBI Bookshelf).
Bhasin S., et al. (2018). Testosterone Therapy in Men With Hypogonadism: An Endocrine Society Clinical Practice Guideline. J Clin Endocrinol Metab, 103 1715-1744.
Boepple P A, Hayes F J, Dwyer A A, Raivio T, Lee H, Crowley W F, Jr. & Pitteloud N 2008 Relative roles of inhibin B and sex steroids in the negative feedback regulation of follicle-stimulating hormone in men across the full spectrum of seminiferous epithelium function. The Journal of clinical endocrinology and metabolism 93 1809-1814.
Boston University Sexual Medicine (accessed 2025). Male Low Testosterone (Low T): prevalence and diagnosis.
Bowen R L & Atwood C S 2004 Living and dying for sex. A theory of aging based on the modulation of cell cycle signaling by reproductive hormones. Gerontology 50 265-290.
Bowen R L, Perry G, Xiong C, Smith M A, Atwood C S 2015 A clinical study of Lupron Depot in the treatment of women with Alzheimer's disease: Preservation of cognitive function in patients taking an acetylcholinesterase inhibitor and treated with high dose Lupron over 48 weeks. Journal of Alzheimer's disease, 44 549-560.
Bowen R L, Verdile G, Liu T, Parlow A F, Perry G, Smith M A, Martins R N & Atwood C S 2004 Luteinizing hormone, a reproductive regulator that modulates the processing of amyloid-beta precursor protein and amyloid-beta deposition. The Journal of biological chemistry 279 20539-20545.
Butler T, Tey S R, Galvin J E, Perry G, Bowen R L, Atwood C S. 2024 Endocrine Dyscrasia in the Etiology and Therapy of Alzheimer's Disease. J Alzheimers Dis 101 705-713.
Byrne J A, Pedersen D A, Clepper L L, Nelson M, Sanger W G, Gokhale S, Wolf D P & Mitalipov S M 2007 Producing primate embryonic stem cells by somatic cell nuclear transfer. Nature 450 497-502.
Cargill S L, Carey J R, Muller H G & Anderson G 2003 Age of ovary determines remaining life expectancy in old ovariectomized mice. Aging Cell 2 185-190.
Carr B R 1998 Williams Textbook of Endocrinology. pp 751-817. Ed F D Wilson J D, Kronenberg H M, Larsen P R. Philadelphia PA: WB Saunders Co.
Cash J N, Angerman E B, Keutmann H T & Thompson T B 2012 Characterization of follistatin-type domains and their contribution to myostatin and activin A antagonism. Molecular endocrinology 26 1167-1178.
Chahal H S & Drake W M 2007 The endocrine system and ageing. The Journal of pathology 211 173-180.
Clark I, Atwood C, Bowen R, Paz-Filho G & Vissel B 2012 Tumor Necrosis Factor-Induced Cerebral Insulin Resistance in Alzheimer's Disease Links Numerous Treatment Rationales. Pharmacological Reviews 64 1004-1026
de Bruin J P, Gosden R G, Finch C E & Leaman B M 2004 Ovarian aging in two species of long-lived rockfish, Sebastes aleutianus and S. alutus. Biol Reprod 71 1036-1042.
DeKretser D M, Hedger M P, Loveland K L & Phillips D J 2002 Inhibins, activins and follistatin in reproduction. Hum. Reprod. Update 8 529-541.
Dreisler E, Poulsen L G, Antonsen S L, Ceausu I, Depypere H, Erel C T, Lambrinoudaki I, Perez-Lopez F R, Simoncini T, Tremollieres F, et al. 2013 EMAS clinical guide: assessment of the endometrium in peri and postmenopausal women. Maturitas 75 181-190.
Dubey A K, Zeleznik A J & Plant T M 1987 In the rhesus monkey (Macaca mulatta), the negative feedback regulation of follicle-stimulating hormone secretion by an action of testicular hormone directly at the level of the anterior pituitary gland cannot be accounted for by either testosterone or estradiol. Endocrinology 121 2229-2237.
Eun Y, Yoo J E, Han K, Kim D, Lee K N, Lee J, Lee D Y, Lee D H, Kim H, Shin D W 2022 Female reproductive factors and risk of joint replacement arthroplasty of the knee and hip due to osteoarthritis in postmenopausal women: a nationwide cohort study of 1.13 million women. Osteoarthritis Cartilage 30 69-80.
Ezashi T, Telugu B P, Alexenko A P, Sachdev S, Sinha S & Roberts R M 2009 Derivation of induced pluripotent stem cells from pig somatic cells. Proceedings of the National Academy of Sciences of the United States of America 106 10993-10998.
Feldman H A, Longcope C, Derby C A, Johannes C B, Araujo A B, Coviello A D, Bremner W J & McKinlay J B 2002 Age trends in the level of serum testosterone and other hormones in middle-aged men: longitudinal results from the Massachusetts male aging study. The Journal of clinical endocrinology and metabolism 87 589-598.
Freeman E W, Sammel M D & Lin H 2009 Temporal associations of hot flashes and depression in the transition to menopause. Menopause 16 728-734.
Freeman E W, Sammel M D, Boorman D W & Zhang R 2014 Longitudinal pattern of depressive symptoms around natural menopause. JAMA psychiatry 71 36-43.
Freeman E W, Sammel M D, Lin H, Gracia C R & Kapoor S 2008 Symptoms in the menopausal transition: hormone and behavioral correlates. Obstetrics and gynecology 111 127-136.
Freeman E W, Sammel M D, Lin H, Gracia C R, Pien G W, Nelson D B & Sheng L 2007 Symptoms associated with menopausal transition and reproductive hormones in midlife women. Obstetrics and gynecology 110 230-240.
Gallego M J, Porayette P, Kaltcheva M M, Bowen R L, Vadakkadath Meethal S & Atwood C S 2010 The pregnancy hormones human chorionic gonadotropin and progesterone induce human embryonic stem cell proliferation and differentiation into neuroectodermal rosettes. Stem cell research & therapy 1 28.
Gallego M J, Porayette P, Kaltcheva M M, Meethal S V & Atwood C S 2009 Opioid and progesterone signaling is obligatory for early human embryogenesis. Stem cells and development 18 737-740.
Gameiro C & Romao F 2010 Changes in the immune system during menopause and aging. Frontiers in bioscience 2 1299-1303.
Gameiro C M, Romao F & Castelo-Branco C 2010 Menopause and aging: changes in the immune system—a review. Maturitas 67 316-320.
Gapstur S M, Gann P H, Kopp P, Colangelo L, Longcope C & Liu K 2002 Serum androgen concentrations in young men: a longitudinal analysis of associations with age, obesity, and race. The CARDIA male hormone study. Cancer epidemiology, biomarkers & prevention: a publication of the American Association for Cancer Research, cosponsored by the American Society of Preventive Oncology 11 1041-1047.
Gharib S D, Wierman M E, Shupnik M A & Chin W W 1990 Molecular biology of the pituitary gonadotropins. Endocrine reviews 11 177-199.
Gleason C E, Cholerton B, Carlsson C M, Johnson S C & Asthana S 2005 Neuroprotective effects of female sex steroids in humans: current controversies and future directions. Cellular and molecular life sciences: CMLS 62 299-312.
Gray P C, Bilizikjian L M & Vale W 2002 Antagonism of activin by inhibin and inhibin receptors: a functional role for betaglycan. Mol. Cell. Endocrinol. 188 254-260.
Hackett G. I. (2024). Long-term cardiovascular safety of testosterone therapy: update post-TRAVERSE. Ther Adv Endocrinol Metab, 15, 204201882412.
Hahn W C & Meyerson M 2001 Telomerase activation, cellular immortalization and cancer. Annals of medicine 33 123-129.
Härkönen K., et al. (2003). The AR CAG repeat and hormonal changes among ageing men. Int J Androl, 26, 187-194.
Harman S. M., et al. (2001). Longitudinal effects of aging on serum total and free testosterone levels in healthy men (BLSA). J Clin Endocrinol Metab, 86, 724-731.
Harman S M, Metter E J, Tobin J D, Pearson J & Blackman M R 2001 Longitudinal effects of aging on serum total and free testosterone levels in healthy men. Baltimore Longitudinal Study of Aging. The Journal of clinical endocrinology and metabolism 86 724-731.
Hayes F J, DeCruz S, Seminara S B, Boepple P A & Crowley W F, Jr. 2001a Differential regulation of gonadotropin secretion by testosterone in the human male: absence of a negative feedback effect of testosterone on follicle-stimulating hormone secretion. The Journal of clinical endocrinology and metabolism 86 53-58.
Hayes F J, Hall J E, Boepple P A & Crowley W F, Jr. 1998 Clinical review 96: Differential control of gonadotropin secretion in the human: endocrine role of inhibin. The Journal of clinical endocrinology and metabolism 83 1835-1841.
Hayes F J, Pitteloud N, DeCruz S, Crowley W F, Jr. & Boepple P A 2001b Importance of inhibin B in the regulation of FSH secretion in the human male. The Journal of clinical endocrinology and metabolism 86 5541-5546.
Hayward C J, Fradette J, Galbraith T, Remy M, Guignard R, Gauvin R, Germain L & Auger F A 2013 Harvesting the potential of the human umbilical cord: isolation and characterisation of four cell types for tissue engineering applications. Cells, tissues, organs 197 37-54.
High circulating follicle-stimulating hormone level is a potential risk factor for renal dysfunction in post-menopausal women. Frontiers in endocrinology (Lausanne) 12, 627903
Hockemeyer D, Soldner F, Cook E G, Gao Q, Mitalipova M & Jaenisch R 2008 A drug-inducible system for direct reprogramming of human somatic cells to pluripotency. Cell stem cell 3 346-353.
Huan Z, Wang Y, Zhang M, Zhang X, Liu Y, Kong L, Xu J 2021 Follicle-stimulating hormone worsens osteoarthritis by causing inflammation and chondrocyte dedifferentiation. FEBS open biology 11 2292-2303.
Illingworth P J, Groome N P, Byrd W, Rainey W E, McNeilly A S, Mather J P & Bremner W J 1996 Inhibin-B: a likely candidate for the physiologically important form of inhibin in men. The Journal of clinical endocrinology and metabolism 81 1321-1325.
Jacobsen M 1991 Histogenesis and morphogenesis of cortical structures. In Developmental neurobiology, pp 401-451. Ed M Jacobsen. New York: Plenum.
Jaffe R B & Keye W R, Jr. 1974 Estradiol augmentation of pituitary responsiveness to gonadotropin-releasing hormone in women. The Journal of clinical endocrinology and metabolism 39 850-855.
Jaffe R B & Keye W R, Jr. 1975 Modulation of pituitary response to hypothalamic releasing factors. Journal of steroid biochemistry 6 1055-1060.
Jaffe R B, Keye W R, Jr. & Young J R 1976 The role of estradiol in modulating LH and FSH response to gonadotropin releasing hormone. Current topics in molecular endocrinology 3 211-254.
Kaufman J. M., T'Sjoen G. G. (2019). Aging and the Male Reproductive System. Endocr Rev, 40, 906-972.
Kaufman J. M., Vermeulen A. (2005). The decline of androgen levels in elderly men and its clinical and therapeutic implications. Endocr Rev, 26(6), 833-876.
Keye W R, Jr. & Jaffe R B 1974 Modulation of pituitary gonadotropin response to gonadotropin-releasing hormone by estradiol. The Journal of clinical endocrinology and metabolism 38 805-810.
Keye W R, Jr. & Jaffe R B 1975 Strength-duration characteristics of estrogen effects on gonadotropin response to gonadotropin-releasing hormone in women. I. Effects of varying duration of estradiol administration. The Journal of clinical endocrinology and metabolism 41 1003-1008.
Keye W R, Jr. & Jaffe R B 1976 Changing patterns of FSH and LH response to gonadotropin-releasing hormone in the puerperium. The Journal of clinical endocrinology and metabolism 42 1133-1138.
Knight P G & Glister C 2001 Potential local regulatory functions of inhibins, activins and follistatin in the ovary. Reproduction 121 503-512.
Kuhbier J W, Weyand B, Radtke C, Vogt P M, Kasper C & Reimers K 2010 Isolation, characterization, differentiation, and application of adipose-derived stem cells. Advances in biochemical engineering/biotechnology 123 55-105.
Lambert-Messerlian G M, Hall J E, Sluss P M, Taylor A E, Martin K A, Groome N P, Crowley W F, Jr. & Schneyer A L 1994 Relatively low levels of dimeric inhibin circulate in men and women with polycystic ovarian syndrome using a specific two-site enzyme-linked immunosorbent assay. The Journal of clinical endocrinology and metabolism 79 45-50.
le Nestour E, Marraoui J, Lahlou N, Roger M, de Ziegler D & Bouchard P 1993 Role of estradiol in the rise in follicle-stimulating hormone levels during the luteal-follicular transition. The Journal of clinical endocrinology and metabolism 77 439-442.
Li Q, Zheng D, Lin H, Zhong F, Liu J, Wu Y, Wang Z, Guan Q, Zhao M, Gao L, Zhao J (2021) Lincoff A. M., et al. (2023). Cardiovascular Safety of Testosterone-Replacement Therapy (TRAVERSE). N Engl J Med, 389, 107-117.
Lincoln G A 2001 The irritable male syndrome. Reproduction, fertility, and development 13 567-576.
Ling N, Ying S Y, Ueno N, Shimasaki S, Esch F, Hotta M & Guillemin R 1986 Pituitary FSH is released by a heterodimer of the beta-subunits from the two forms of inhibin. Nature 321 779-782.
Liu J H & Yen S S 1983 Induction of midcycle gonadotropin surge by ovarian steroids in women: a critical evaluation. The Journal of clinical endocrinology and metabolism 57 797-802.
Liu P Y, Beilin J, Meier C, Nguyen T V, Center J R, Leedman P J, Seibel M J, Eisman J A & Handelsman D J 2007 Age-related changes in serum testosterone and sex hormone binding globulin in Australian men: longitudinal analyses of two geographically separate regional cohorts. The Journal of clinical endocrinology and metabolism 92 3599-3603.
Liu Y, Zhang M, Kong D, Wang Y, Li J, Liu W, Fu Y, Xu J. 2020 High follicle-stimulating hormone levels accelerate cartilage damage of knee osteoarthritis in postmenopausal women through the PI3K/AKT/NF-kappaB pathway. FEBS Open Bio. 10 2235-2245.
Lizneva D, Yuen T, Sun L, Kim S M, Atabiekov I, Munshi L B, Epstein S, New M, Zaidi M 2018 Matrix Biol. 71-72 70-81.
Llaneza P, Fernandez-Inarrea J M, Arnott B, Garcia-Portilla M P, Chedraui P & Perez-Lopez F R 2011 Sexual function assessment in postmenopausal women with the 14-item changes in sexual functioning questionnaire. The journal of sexual medicine 8 2144-2151.
Llaneza P, Garcia-Portilla M P, Llaneza-Suarez D, Armott B & Perez-Lopez F R 2012 Depressive disorders and the menopause transition. Maturitas 71 120-130.
Lo K C, Lei Z, Rao Ch V, Beck J & Lamb D J 2004 De novo testosterone production in luteinizing hormone receptor knockout mice after transplantation of leydig stem cells. Endocrinology 145 4011-4015.
Ludwig T E, Bergendahl V, Levenstein M E, Yu J, Probasco M D & Thomson J A 2006 Feeder-independent culture of human embryonic stem cells. Nature methods 3 637-646.
Lusis A J 2000 Atherosclerosis. Nature 407 233-241.
Malgieri A, Kantzari E, Patrizi M P & Gambardella S 2010 Bone marrow and umbilical cord blood human mesenchymal stem cells: state of the art. International journal of clinical and experimental medicine 3 248-269.
Manini I, Gulino L, Gava B, Pierantozzi E, Curina C, Rossi D, Brafa A, D'Aniello C & Sorrentino V 2011 Multi-potent progenitors in freshly isolated and cultured human mesenchymal stem cells: a comparison between adipose and dermal tissue. Cell and tissue research 344 85-95.
Marynick S P, Loriaux D L, Sherins R J, Pita J C, Jr. & Lipsett M B 1979 Evidence that testosterone can suppress pituitary gonadotropin secretion independently of peripheral aromatization. The Journal of clinical endocrinology and metabolism 49 396-398.
Mason J B, Cargill S L, Anderson G B & Carey J R 2009 Transplantation of young ovaries to old mice increased life span in transplant recipients. J Gerontol A Biol Sci Med Sci 64 1207-1211.
Masterson J. M., et al. (2019). Elevated Body Mass Index Is Associated with Secondary Hypogonadism among Men Presenting to a Tertiary Academic Medical Center. World Journal of Men's Health, 37(1), 93-98.
Matzkin M. E., et al. (2021). Hallmarks of Testicular Aging. Cells, 10, 3114.
Matzuk M. M., et al. (1992). Alpha-inhibin is a tumor-suppressor gene with gonadal specificity in mice. Nature. 360, 313-319.
McPhie D L, Coopersmith R, Hines-Peralta A, Chen Y, Ivins K J, Manly S P, Kozlowski M R, Neve K A & Neve R L 2003 DNA synthesis and neuronal apoptosis caused by familial Alzheimer disease mutants of the amyloid precursor protein are mediated by the p21 activated kinase PAK3. The Journal of neuroscience: the official journal of the Society for Neuroscience 23 6914-6927.
Medscape (2019). Klinefelter Syndrome—Epidemiology (≈1:660 males in the U.S.).
Meethal S V, Liu T, Chan H W, Ginsburg E, Wilson A C, Gray D N, Bowen R L, Vonderhaar B K & Atwood C S 2009 Identification of a regulatory loop for the synthesis of neurosteroids: a steroidogenic acute regulatory protein-dependent mechanism involving hypothalamic-pituitary-gonadal axis receptors. J Neurochem 110 1014-1027.
Meethal S V, Smith M A, Bowen R L & Atwood C S 2005 The gonadotropin connection in Alzheimer's disease. Endocrine 26 317-326.
Messinis I E, Messini C I, Anifandis G, Garas A, Daponte A 2018 Gonadotropin surge-attenuating factor: A nonsteroidal ovarian hormone controlling GnRH-induced LH secretion in the normal menstrual cycle. Vitamins and Hormones 107 263-286.
Monterrosa-Castro A, Marrugo-Florez M, Romero-Perez I, Chedraui P, Fernandez-Alonso A M & Perez-Lopez F R 2013 Prevalence of insomnia and related factors in a large mid-aged female Colombian sample. Maturitas 74 346-351.
Monterrosa-Castro A, Romero-Perez I, Marrugo-Florez M, Fernandez-Alonso A M, Chedraui P & Perez-Lopez F R 2012 Quality of life in a large cohort of mid-aged Colombian women assessed using the Cervantes Scale. Menopause 19 924-930.
Multani A S, Ozen M, Narayan S, Kumar V, Chandra J, McConkey D J, Newman R A & Pathak S 2000 Caspase-dependent apoptosis induced by telomere cleavage and TRF2 loss. Neoplasia 2 339-345. Nature, 360, 313-319.
Neaves W. B., et al. (1985). Age-related change in numbers of interstitial cells (including Leydig cells) in human testis. Biol Reprod, 33, 935-942.
Nie X., et al. (2022). Single-cell analysis of human testis aging and correlations with pathology. Dev Cell, 57, 2363-2376.
Ongaro L, Zhou X, Wang Y, Schultz H, Zhou Z, Buddle E R S, Brûlé E, Lin Y F, Schang G, Hagg A, Castonguay R, Liu Y, Su G H, Seidah N G, Ray K C, Karp S J, Boehm U, Ruf-Zamojski F, Sealfon S C, Walton K L, Lee S J, Bernard D J. (2025) Muscle-derived myostatin is a major endocrine driver of follicle-stimulating hormone synthesis. Science 387 329-336.
Ornat L, Martinez-Dearth R, Munoz A, Franco P, Alonso B, Tajada M & Perez-Lopez F R 2013 Sexual function, satisfaction with life and menopausal symptoms in middle-aged women. Maturitas 75 261-269.
Ossewaarde M E, Bots M L, Verbeek A L, Peeters P H, van der Graaf Y, Grobbee D E & van der Schouw Y T 2005 Age at menopause, cause-specific mortality and total life expectancy. Epidemiology 16 556-562.
Paganini-Hill A, Corrada M M & Kawas C H 2006 Increased longevity in older users of postmenopausal estrogen therapy: the Leisure World Cohort Study. Menopause 13 12-18.
Paniagua R., et al. (1987). Seminiferous tubule involution in elderly men. Biol Reprod, 36-939-955.
Parker W H & Manson J E 2009 Oophorectomy and cardiovascular mortality: is there a link? Menopause 16 1-2.
Patel A. S., et al. (2018). Testosterone is a contraceptive and should not be used in men who desire fertility. World J Mens Health 36 103-115.
Pendergraft S. S., et al. (2017). Human testis organoids form in vitro and produce key testicular hormones. Sci Rep 7 5266.
Perez-Lopez F R, Fernandez-Alonso A M, Trabalon-Pastor M, Vara C & Chedraui P 2012 Assessment of sexual function and related factors in mid-aged sexually active Spanish women with the six-item Female Sex Function Index. Menopause 19 1224-1230.
Pien G W, Sammel M D, Freeman E W, Lin H & DeBlasis T L 2008 Predictors of sleep quality in women in the menopausal transition. Sleep 31 991-999.
Pitteloud N, Dwyer A A, DeCruz S, Lee H, Boepple P A, Crowley W F, Jr. & Hayes F J 2008a Inhibition of luteinizing hormone secretion by testosterone in men requires aromatization for its pituitary but not its hypothalamic effects: evidence from the tandem study of normal and gonadotropin-releasing hormone-deficient men. The Journal of clinical endocrinology and metabolism 93 784-791.
Pitteloud N, Dwyer A A, DeCruz S, Lee H, Boepple P A, Crowley W F, Jr. & Hayes F J 2008b The relative role of gonadal sex steroids and gonadotropin-releasing hormone pulse frequency in the regulation of follicle-stimulating hormone secretion in men. The Journal of clinical endocrinology and metabolism 93 2686-2692.
Ponce O. J., et al. (2018). The Efficacy and Adverse Events of Testosterone Replacement Therapy: A Systematic Review and Meta-analysis. J Clin Endocrinol Metab, 103(5), 1745-1754.
Porayette P, Gallego M J, Kaltcheva M M, Bowen R L, Vadakkadath Meethal S & Atwood C S 2009 Differential processing of amyloid-beta precursor protein directs human embryonic stem cell proliferation and differentiation into neuronal precursor cells. The Journal of biological chemistry 284 23806-23817.
Rastrelli G., et al. (2015). Development of and Recovery from Secondary Hypogonadism in Aging Men: Prospective EMAS Results. The Journal of Clinical Endocrinology & Metabolism 100 3172-3181.
Ratajczak M Z, Zuba-Surma E K, Wojakowski W, Ratajczak J & Kucia M 2008 Bone Marrow—Home of Versatile Stem Cells. Transfusion medicine and hemotherapy: offizielles Organ der Deutschen Gesellschaft fur Transfusionsmedizin und Immunhamatologie 35 248-259.
Reichlin S 1998 Williams Textbook of Endocrinology. In Williams Textbook of Endocrinology. 10 ed, pp 165-248. Eds J D Wilson, D W Foster, H M Kronenberg & P R Larsen. Philadelphia PA: WB Saunders Co.
Rocca W A, Grossardt B R, de Andrade M, Malkasian G D & Melton L J, 3rd 2006 Survival patterns after oophorectomy in premenopausal women: a population-based cohort study. The lancet oncology 7 821-828.
Rocca W A, Grossardt B R, Geda Y E, Gostout B S, Bower J H, Maraganore D M, de Andrade M & Melton L J, 3rd 2008 Long-term risk of depressive and anxiety symptoms after early bilateral oophorectomy. Menopause 15 1050-1059.
Rocca W A, Grossardt B R, Miller V M, Shuster L T & Brown R D, Jr. 2012 Premature menopause or early menopause and risk of ischemic stroke. Menopause 19 272-277.
Rosario E R, Chang L, Head E H, Stanczyk F Z & Pike C J 2011 Brain levels of sex steroid hormones in men and women during normal aging and in Alzheimer's disease. Neurobiology of Aging 32 604-613.
Rosario E R, Chang L, Stanczyk F Z & Pike C J 2004 Age-related testosterone depletion and the development of Alzheimer disease. JAMA: the journal of the American Medical Association 292 1431-1432.
Santen R J 1975 Is aromatization of testosterone to estradiol required for inhibition of luteinizing hormone secretion in men? The Journal of clinical investigation 56 1555-1563.
Schnorr J A, Bray M J & Veldhuis J D 2001 Aromatization mediates testosterone's short-term feedback restraint of 24-hour endogenously driven and acute exogenous gonadotropin-releasing hormone-stimulated luteinizing hormone and follicle-stimulating hormone secretion in young men. The Journal of clinical endocrinology and metabolism 86 2600-2606.
Sherins R J & Loriaux D L 1973 Studies of the role of sex steroids in the feedback control of FSH concentrations in men. The Journal of clinical endocrinology and metabolism 36 886-893.
Simon A F, Shih C, Mack A & Benzer S 2003 Steroid control of longevity in Drosophila melanogaster. Science 299 1407-1410.
Sittadjody S, Saul J M, Joo S, Yoo J J, Atala A & Opara E C 2013 Engineered multilayer ovarian tissue that secretes sex steroids and peptide hormones in response to gonadotropins. Biomaterials 34 2412-2420.
Snyder P. J., et al. (2016). Effects of Testosterone Treatment in Older Men (Sexual Function, Physical Function, Vitality). N Engl J Med, 374 611-624.
Snyder P. J., et al. (2017). Effect of Testosterone on Volumetric Bone Density and Strength (Bone Trial). JAMA Intern Med, 177 471-479.
Steiner R A, Bremner W J & Clifton D K 1982 Regulation of luteinizing hormone pulse frequency and amplitude by testosterone in the adult male rat. Endocrinology 111 2055-2061.
Sternbach H 1998 Age-associated testosterone decline in men: clinical issues for psychiatry. The American journal of psychiatry 155 1310-1318.
Sun L, Peng Y, Sharrow A C, Iqbal J, Zhang Z, Papachristou D J, Zaidi S, Zhu L L, Yaroslavskiy B B, Zhou H, et al. 2006 FSH directly regulates bone mass. Cell 125 247-260.
Tajar A., et al. (2010). Hypogonadism in Aging Men: Evidence from EMAS. J Clin Endocrinol Metab 95 1810-1818.
Taylor A E, Whitney H, Hall J E, Martin K & Crowley W F, Jr. 1995 Midcycle levels of sex steroids are sufficient to recreate the follicle-stimulating hormone but not the luteinizing hormone midcycle surge: evidence for the contribution of other ovarian factors to the surge in normal women. The Journal of clinical endocrinology and metabolism 80 1541-1547.
Tholpady S S, Katz A J & Ogle R C 2003 Mesenchymal stem cells from rat visceral fat exhibit multipotential differentiation in vitro. The anatomical record. Part A, Discoveries in molecular, cellular, and evolutionary biology 272 398-402.
Thorner M, Vance M, Laws Jr. E, Horvath E & Kovacs K 1998 Williams Textbook of Endocrinology. pp 249-340. Ed FD Wilson J D, Kronenberg H M, Larsen P R. Philadelphia PA: WB Saunders Co.
Travison T G, Araujo A B, Kupelian V, O'Donnell A B & McKinlay J B 2007a The relative contributions of aging, health, and lifestyle factors to serum testosterone decline in men. The Journal of clinical endocrinology and metabolism 92 549-555.
Travison T G, Araujo A B, O'Donnell A B, Kupelian V & McKinlay J B 2007b A population-level decline in serum testosterone levels in American men. The Journal of clinical endocrinology and metabolism 92 196-202.
Tserotas K & Merino G 1998 Andropause and the aging male. Archives of andrology 40 87-93.
T'Sjoen G G, De Vos S, Goemaere S, Van Pottelbergh I, Dierick M, Van Heeringen C & Kaufman J M 2005 Sex steroid level, androgen receptor polymorphism, and depressive symptoms in healthy elderly men. Journal of the American Geriatrics Society 53 636-642.
Tuli R, Seghatoleslami M R, Tuli S, Wang M L, Hozack W J, Manner P A, Danielson K G & Tuan R S 2003a A simple, high-yield method for obtaining multipotential mesenchymal progenitor cells from trabecular bone. Molecular biotechnology 23 37-49.
Tuli R, Tuli S, Nandi S, Wang M L, Alexander P G, Haleem-Smith H, Hozack W J, Manner P A, Danielson K G & Tuan R S 2003b Characterization of multipotential mesenchymal progenitor cells derived from human trabecular bone. Stem Cells 21 681-693.
Vadakkadath Meethal S & Atwood C S 2005 The role of hypothalamic-pituitary-gonadal hormones in the normal structure and functioning of the brain. Cell Mol Life Sci 62 257-270.
Vadakkadath Meethal S, Gallego M J, Haasl R J, Petras S J, 3rd, Sgro J Y & Atwood C S 2006 Identification of a gonadotropin-releasing hormone receptor orthologue in Caenorhabditis elegans. BMC evolutionary biology 6 103.
Vale W, Rivier J, Vaughn J, McClintock R, Corrigan A, Woo W, Darr D & Spiess J 1986 Purification and characterization of an FSH releasing protein from porcine ovarian follicular fluid. Nature 321 776-779.
Veldhuis J D, Urban R J & Dufau M L 1992 Evidence that androgen negative feedback regulates hypothalamic gonadotropin-releasing hormone impulse strength and the burst-like secretion of biologically active luteinizing hormone in men. The Journal of clinical endocrinology and metabolism 74 1227-1235.
Wallace W H & Kelsey T W 2010 Human ovarian reserve from conception to the menopause. PLoS One 5 e8772.
Welt C K, Martin K A, Taylor A E, Lambert-Messerlian G M, Crowley W F, Jr., Smith J A, Schoenfeld D A & Hall J E 1997 Frequency modulation of follicle-stimulating hormone (FSH) during the luteal-follicular transition: evidence for FSH control of inhibin B in normal women. The Journal of clinical endocrinology and metabolism 82 2645-2652.
Wilson A C, Clemente L, Liu T, Bowen R L, Meethal S V & Atwood C S 2008 Reproductive hormones regulate the selective permeability of the blood-brain barrier. Biochim Biophys Acta 1782 401-407.
Wilson A C, Salamat M S, Haasl R J, Roche K M, Karande A, Meethal S V, Terasawa E, Bowen R L & Atwood C S 2006 Human neurons express type I GnRH receptor and respond to GnRH I by increasing luteinizing hormone expression. The Journal of endocrinology 191 651-663.
Winters S J, Janick J J, Loriaux D L & Sherins R J 1979a Studies on the role of sex steroids in the feedback control of gonadotropin concentrations in men. II. Use of the estrogen antagonist, clomiphene citrate. The Journal of clinical endocrinology and metabolism 48 222-227.
Winters S J, Sherins R J & Loriaux D L 1979b Studies on the role of sex steroids in the feedback control of gonadotropin concentrations in men. III. Androgen resistance in primary gonadal failure. The Journal of clinical endocrinology and metabolism 48 553-558.
Xiong J, Kang S S, Wang Z, Liu X, Kuo T C, Korkmaz F, Padilla A, Miyashita S, Chan P, Zhang Z, Katsel P, Burgess J, Gumerova A, Ievleva K, Sant D, Yu S P, Muradova V, Frolinger T, Lizneva D, Iqbal J, Goosens K A, Gera S, Rosen C J, Haroutunian V, Ryu V, Yuen T, Zaidi M, Ye K 2022 FSH blockade improves cognition in mice with Alzheimer's disease Nature 603 470-476.
Yazawa T, Mizutani T, Yamada K, Kawata H, Sekiguchi T, Yoshino M, Kajitani T, Shou Z, Umezawa A & Miyamoto K 2006 Differentiation of adult stem cells derived from bone marrow stroma into Leydig or adrenocortical cells. Endocrinology 147 4104-4111.
Ying S Y 1988 Inhibins, activins, and follistatins: gonadal proteins modulating the secretion of follicle-stimulating hormone. Endocrine reviews 9 267-293.
Yonker J A, Chang V, Roetker N S, Hauser T S, Hauser R M & Atwood C S 2011 Hypothalamic-pituitary-gonadal axis homeostasis predicts longevity. AGE.
Young J R & Jaffe R B 1976 Strength-duration characteristics of estrogen effects on gonadotropin response to gonadotropin-releasing hormone in women. II. Effects of varying concentrations of estradiol. The Journal of clinical endocrinology and metabolism 42 432-442.
Yu J, Hu K, Smuga-Otto K, Tian S, Stewart R, Slukvin, II & Thomson J A 2009 Human induced pluripotent stem cells free of vector and transgene sequences. Science 324 797-801.
Yue X, Lu M, Lancaster T, Cao P, Honda S, Staufenbiel M, Harada N, Zhong Z, Shen Y & Li R 2005 Brain estrogen deficiency accelerates Abeta plaque formation in an Alzheimer's disease animal model. Proc Natl Acad Sci U S A 102 19198-19203.
Zhu M, Heydarkhan-Hagvall S, Hedrick M, Benhaim P & Zuk P 2013 Manual isolation of adipose-derived stem cells from human lipoaspirates. Journal of visualized experiments: JoVE e50585.
Zuk P A, Zhu M, Mizuno H, Huang J, Futrell J W, Katz A J, Benhaim P, Lorenz H P & Hedrick M H 2001 Multilineage cells from human adipose tissue: implications for cell-based therapies. Tissue engineering 7 211-228.

What is claimed is:

1. A method of treating male hypogonadism in a subject in need thereof comprising administering to the subject testicular organoids generated from isolated male progenitor cells.

2. The method of claim 1, wherein the subject is not being administered testosterone replacement therapy.

3. The method of claim 1, wherein the hypogonadism is a result of andropause or testosterone deficiency syndrome (TDS), or congenital or acquired primary hypogonadism.

4. The method of claim 1, wherein the subject is a male mammal.

5. The method of claim 4 wherein the subject is a human.

6. The method of claim 5, wherein the human is 50 years old or older.

7. The method of claim 1, wherein method comprises injection of the organoids.

8. The method of claim 1, wherein the male progenitor cells are isolated from the subject.

9. The method of claim 1, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

10. The method of claim 1, wherein the method restores hormone balance.

11. The method of claim 1, wherein the method does not further comprise pre-treating the subject with a chorionic gonadotrophin (CG).

12. The method of claim 1, wherein the method further comprises generating the testicular organoids by expanding isolated male progenitor cells, generating a spheroid under dynamic culture conditions, and culturing the spheroid under dynamic culture conditions to generate the testicular organoid.

13. The method of claim 12, wherein the method comprises culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

14. The method of claim 1, wherein the progenitor cells are adult-derived or umbilical cord-derived mesenchymal stem cells, or adipose-derived stem cells (ADSC).

15. A method of generating a testicular organoid, the method comprising:
(a) generating a spheroid from isolated male progenitor cells under dynamic conditions; and
(b) culturing the spheroid under dynamic conditions in a culture medium supplemented with a luteinizing hormone (LH), follicle stimulating hormone (FSH), a thyroid hormone, human insulin-like growth factor 1 (IGF-1), human glial cell line-derived neurotrophic factor (GDNF), retinoic acid, smoothened agonist (SAG), 8-bromoadenosine 3′,5′-cyclic adenosine monophosphate (8-Br-cAMP), testosterone, and platelet-derived growth factor AA or platelet-derived growth factor AB to generate the testicular organoid.

16. The method of claim 15, wherein the testicular organoid comprises cells expressing GFRA1, VEGF, VEGFR2, PDGF-Rα, Cyp11A1, 3β-HSD, 17β-HSD, LHCGR, α-SMA, CD11b, MHC CII, CD64, CD95, Sox9, AR, inhibin β-B, and FSHR.

17. The method of claim 16, wherein the testicular organoid comprises at least 5-30% cells expressing PDGF-Rα, Cyp11A1, 3β-HSD, 17β-HSD, and LHCGR, about 5-50% of cells expressing α-SMA and CD11b, about 5-50% of cells expressing MHC CII and CD64, at least 5-30% of cells expressing Sox9, AR, inhibin β-B, and FSHR, and about 5-30% of cells expressing VEGF and VEGFR2, and cells expressing about 3-15% GFRA1.

18. The method of claim 15, wherein the progenitor cells are adult-derived or umbilical cord-derived mesenchymal stem cells, or adipose-derived stem cells (ADSC).

19. The method of claim 18, wherein the method comprises culturing the cells for at least 7 days or for about 14 days to about 27 days.

20. A method of increasing health span or longevity in a male subject in need thereof comprising administering to the subject testicular organoids generated from isolated male progenitor cells.

21. The method of claim 20, wherein the male subject is a male mammal or human.

22. The method of claim 20, wherein method comprises injection of the organoids.

23. The method of claim 20, wherein the method does not further comprise pre-treating the subject with a chorionic gonadotrophin (CG).

24. A method of rebalancing the hypothalamic-pituitary-gonadal (HPG) axis in a male subject in need thereof comprising administering to the subject testicular organoids generated from isolated male progenitor cells.

25. The method of claim 24, wherein the male subject is a male mammal or human.

26. The method of claim 24, wherein method comprises injection of the organoids.

27. The method of claim 24, wherein the method does not further comprise pre-treating the subject with a chorionic gonadotrophin (CG).

28. The method of claim 24, wherein the subject is suffering from an age-related disease.

29. A method of slowing or halting diseases of aging in a male subject in need thereof comprising administering to the subject testicular organoids generated from isolated male progenitor cells.

30. The method of claim 29, wherein the male subject is a male mammal or human.

31. The method of claim 29, wherein method comprises injection of the organoids.

32. The method of claim 29, wherein the method does not further comprise pre-treating the subject with a chorionic gonadotrophin (CG).
